SEQUENCE LISTING (3 sequences)

SEQ ID NO: 1 - DNA, 8543 bp, Homo sapiens
cacctacctccccgccgctccagagggggctcgcagagctgaggacgcgcgcagcgctgctcaaggtctctctctctcagcaccctcgccggccggcgtctgacgcgggtgccagggtctccgggcacctttcagtgtccattccctcagccagccaggactccgcaacccagcagttgccgctgcggccacagcccgaggggacctgcggacaggacgccggcaggaggaggggtgcgcagcgcccgcgcagagcgtctccctcgctacgcagcgagacccgggcctcccggccccaggagcccccagctgcctcgccaggtgtgtgggactgaagttcttggagaagggagtccaactcttcaaggtgaactatgaccactttactctgggttttcgtgactctgagggtcatcactgcagctgtcactgtagaaacttcagaccatgacaactcgctgagtgtcagcatcccccaaccgtccccgctgagggtcctcctggggacctccctcaccatcccctgctatttcatcgaccccatgcaccctgtgaccaccgccccttctaccgccccactggccccaagaatcaagtggagccgtgtgtccaaggagaaggaggtagtgctgctggtggccactgaagggcgcgtgcgggtcaacagtgcctatcaggacaaggtctcactgcccaactacccggccatccccagtgacgccaccttggaagtccagagcctgcgctccaatgactctggggtctaccgctgcgaggtgatgcatggcatcgaggacagcgaggccaccctggaagtcgtggtgaaaggcatcgtgttccattacagagccatctctacacgctacaccctcgactttgacagggcgcagcgggcctgcctgcagaacagtgccatcattgccacgcctgagcagctgcaggccgcctacgaagacggcttccaccagtgtgacgccggctggctggctgaccagactgtcagataccccatccacactccccgggaaggctgctatggagacaaggatgagtttcctggtgtgaggacgtatggcatccgagacaccaacgagacctatgatgtgtactgcttcgccgaggagatggagggtgaggtcttttatgcaacatctccagagaagttcaccttccaggaagcagccaatgagtgccggcggctgggtgcccggctggccaccacgggccagctctacctggcctggcaggctggcatggacatgtgcagcgccggctggctggccgaccgcagcgtgcgctaccccatctccaaggcccggcccaactgcggtggcaacctcctgggcgtgaggaccgtctacgtgcatgccaaccagacgggctaccccgacccctcatcccgctacgacgccatctgctacacaggtgaagactttgtggacatcccagaaaacttctttggagtggggggtgaggaggacatcaccgtccagacagtgacctggcctgacatggagctgccactgcctcgaaacatcactgagggtgaagcccgaggcagcgtgatccttaccgtaaagcccatcttcgaggtctcccccagtcccctggaacccgaggagcccttcacgtttgcccctgaaataggggccactgccttcgctgaggttgagaatgagactggagaggccaccaggccctggggctttcccacacctggcctgggccctgccacggcattcaccagtgaggacctcgtcgtgcaggtgaccgctgtccctgggcagccgcatttgccagggggggtcgtcttccactaccgcccgggacccacccgctactcgctgacctttgaggaggcacagcaggcctgcctgcgcacgggggcggtcattgcctcgccggagcagctccaggccgcctacgaagcaggctatgagcagtgtgacgccggctggctgcgggaccagaccgtcagataccccattgtgagcccccggaccccatgcgtgggtgacaaggacagcagcccaggggtcaggacctatggcgtgcgcccatcaacagagacctacgatgtctactgctttgtagacagacttgagggggaggtgttcttcgccacacgccttgagcagttcaccttccaggaagcactggagttctgtgaatctcacaatgctacgctggccaccacgggccagctctacgccgcctggagccgcggcctggacaagtgctatgccggctggctggccgacggcagcctccgctaccccatcgtcaccccaaggcctgcctgcggtggggacaagccaggcgtgagaacggtctacctctaccctaaccagacgggcctcccagacccactgtcccggcaccatgccttctgcttccgaggcatttcagcggttccttctccaggagaagaagagggtggcacacccacatcaccctctggtgtggaggagtggatcgtgacccaagtggttcctggtgtggctgctgtccccgtagaagaggagacaactgctgtaccctcaggggagactactgccatcctagagttcaccaccgagccagaaaaccagacagaatgggaaccagcctataccccagtgggcacatccccgctgccagggatccttcctacttggcctcccactggcgcagcaacagaggaaagtacagaaggcccttctgcaactgaagtgccctctgcctcagaggaaccatccccctcagaggtgccattcccctcagaggagccatccccctcagaggaaccattcccctcagtgaggccattcccctcagtggagctgttcccctcagaggagccattcccctccaaggagccatccccctcagaggaaccatcagcctcggaagagccgtatacaccttcaccccccgtgcccagctggactgagctgcccagctctggggaggaatctggggcccctgatgtcagtggtgacttcacaggcagtggagatgtttcaggacaccttgacttcagtgggcagctgtcaggggacagggcaagtggactgccctctggagacctggactccagtggtcttacttccacagtgggctcaggcctgcctgtggaaagtggactaccctcaggggatgaagagagaattgagtggcccagcactcctacggttggtgaactgccctctggagctgagatcctagagggctctgcctctggagttggggatctcagtggacttccttctggagaagttctagagacctctgcctctggagtaggagacctcagtgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactactgcccctggagtagaggagatcagcgggcttccttctggagaagttctagagactactgcccctggagtagatgagatcagtgggcttccttctggagaagttctagagactactgcccctggagtagaggagatcagcgggcttccttctggagaagttctagagacttctacctctgcggtaggggacctcagtggacttccttctggaggagaagttctagagatttctgtctctggagtagaggacatcagtgggcttccttctggagaggttgtagagacttctgcctctggaatagaggatgtcagtgaacttccttcaggagaaggtctagagacctctgcttctggagtagaggacctcagcaggctcccttctggagaagaagttctagagatttctgcctctggatttggggacctcagtggacttccttctggaggagaaggtctagagacctctgcttctgaagtagggactgacctcagtgggcttccttctggaagggagggtctagagacttcagcttctggagctgaggacctcagtgggttgccttctggaaaagaagacttggtggggtcagcttctggagacttggacttgggcaaactgccttctggaactctaggaagtgggcaagctccagaaacaagtggtcttccctctggatttagtggtgagtattctggggtggaccttggaagtggcccaccctctggcctgcctgactttagtggacttccatctggattcccaactgtttccctagtggattctacattggtggaagtggtcacagcctccactgcaagtgaactggaagggaggggaaccattggcatcagtggtgcaggagaaatatctggactgccctccagtgagctggacattagtgggagagctagtggactcccttcaggaactgaactcagtggccaagcatctgggtctcctgatgtcagtggggaaatacctggactctttggtgtcagtggacagccatcagggtttcctgacactagtggggaaacatctggagtgactgagcttagcgggctgtcctctggacaaccaggtattagtggagaagcatctggagttctttatggcactagtcaaccctttggcataactgatctgagtggagaaacatctggggtccctgatctcagtgggcagccttcagggttaccagggttcagtggggcaacatcaggagtccctgacctggtttctggtaccacgagtggcagcggtgaatcttctgggattacatttgtggacaccagtttggttgaagtggcccctactacatttaaagaagaagaaggcttagggtctgtggaactcagtggcctcccttccggagaggcagatctgtcaggcaaatctgggatggtggatgtcagtggacagttttctggaacagtcgattccagtgggtttacatcccagactccggaattcagtggcctaccaagtggcatagctgaggtcagtggagaatcctccagagctgagattgggagcagcctgccctcgggagcatattatggcagtggaactccatctagtttccccactgtctctcttgtagacagaactttggtggaatctgtaacccaggctccaacagcccaagaggcaggagaagggccttctggcattttagaactcagtggtgctcattctggagcaccagacatgtctggggagcattctggatttctggacctaagtgggctgcagtccgggctgatagagcccagcggagagccaccaggtactccatattttagtggggattttgccagcaccaccaatgtaagtggagaatcctctgtagccatgggcaccagtggagaggcctcaggacttccagaagttactttaatcacttctgagttcgtggagggtgttactgaaccaactatttctcaggaactaggccaaaggccccctgtgacacacacaccccagctttttgagtccagtggaaaagtctccacagctggggacattagtggagctaccccagtgctccctgggtctggagtagaagtatcatcagtcccagaatctagcagtgagacgtccgcctatcctgaagctgggttcggggcatctgccgcccctgaggccagcagagaagattctgggtcccctgatctgagtgaaaccacctctgcattccacgaagctaaccttgagagatcctctggcctaggagtgagcggcagcactttgacatttcaagaaggcgaggcgtccgctgccccagaagtgagtggagaatccaccaccaccagtgatgtggggacagaggcaccaggcttgccttcagccactcccacggcttctggagacaggactgaaatcagcggagacctgtctggtcacacctcgcagctgggcgttgtcatcagcaccagcatcccagagtctgagtggacccagcagacccagcgccctgcagagacgcatctagaaattgagtcctcaagcctcctgtactcaggagaagagactcacacagtcgaaacagccacctccccaacagatgcttccatcccagcttctccggaatggaaacgtgaatcagaatcaactgctgcagaccaggaggtatgtgaggagggctggaacaagtaccagggccactgttaccgccacttcccggaccgcgagacctgggtggatgctgagcgccggtgtcgggagcagcagtcacacctgagcagcatcgtcacccccgaggagcaggagtttgtcaacaacaatgcccaagactaccagtggatcggcctgaacgacaggaccatcgaaggggacttccgctggtcagatggacaccccatgcaatttgagaactggcgccccaaccagcctgacaacttttttgccgctggagaggactgtgtggtgatgatctggcacgagaagggcgagtggaatgatgttccctgcaattaccacctccccttcacgtgtaaaaagggcacagccaccacctacaaacgcagactacagaagcggagctcacggcaccctcggaggagccgccccagcacagcccactgagaagagcttccaggacgcacccaggacgctgagcccaggagcctgccaggctgacgtgcatcccacccagacggtgtcctcttcttgtcgctttttgtcatataaggaatcccattaaagaaggaaaaaaataaatcccacatttgtgtatgcacccactcacccctccaaatcagcaaaaccgcatctaatttgtccgccgaatgccaaagcaaagcaaacttattataacccttggactgagtttagagacatttcttcaatttcccatcgtgcctttccagggaccagtgcagggacagggggagaaggggaggggttaagttaaataaagaagattatttttgtttcctgactttatccaagagcagtgcaatcgttggttatttcacctccagggagagctagggaggagggaggagggctccaaaggagctggaaggagcagaggcctgagagcaggaagaactcggaaccgcagctgaatgtattggatgagaaggagccaggagggctacaccatctgtatgagggaaaagccttgggggagaggggtgggttcctgcctcctgccgagggtaagccggcaggagagagccatcagagggacctccgctgcctgggagttgggttccctccaagggtccctctttcagtgtcctctctctcacctgggtctgccaccctaacaggtggcaactcggcagggctgctgggggcacttcctgcccagtggggggtgccgcccaaccttctcccctccccacccccgcccccgggaccgtgcaggcaccagggttccgtgcacctatttatatttttgaaaactgaagattataatattataataataataaagacattggaagagat

SEQ ID NO: 2 - DNA, 8840 bp, Homo sapiens
cacctacctccccgccgctccagagggggctcgcagagctgaggacgcgcgcagcgctgctcaaggtctctctctctcagcaccctcgccggccggcgtctgacgcgggtgccagggtctccgggcacctttcagtgtccattccctcagccagccaggactccgcaacccagcagttgccgctgcggccacagcccgaggggacctgcggacaggacgccggcaggaggaggggtgcgcagcgcccgcgcagagcgtctccctcgctacgcagcgagacccgggcctcccggccccaggagcccccagctgcctcgccaggtgtgtgggactgaagttcttggagaagggagtccaactcttcaaggtgaactatgaccactttactctgggttttcgtgactctgagggtcatcactgcagctgtcactgtagaaacttcagaccatgacaactcgctgagtgtcagcatcccccaaccgtccccgctgagggtcctcctggggacctccctcaccatcccctgctatttcatcgaccccatgcaccctgtgaccaccgccccttctaccgccccactggccccaagaatcaagtggagccgtgtgtccaaggagaaggaggtagtgctgctggtggccactgaagggcgcgtgcgggtcaacagtgcctatcaggacaaggtctcactgcccaactacccggccatccccagtgacgccaccttggaagtccagagcctgcgctccaatgactctggggtctaccgctgcgaggtgatgcatggcatcgaggacagcgaggccaccctggaagtcgtggtgaaaggcatcgtgttccattacagagccatctctacacgctacaccctcgactttgacagggcgcagcgggcctgcctgcagaacagtgccatcattgccacgcctgagcagctgcaggccgcctacgaagacggcttccaccagtgtgacgccggctggctggctgaccagactgtcagataccccatccacactccccgggaaggctgctatggagacaaggatgagtttcctggtgtgaggacgtatggcatccgagacaccaacgagacctatgatgtgtactgcttcgccgaggagatggagggtgaggtcttttatgcaacatctccagagaagttcaccttccaggaagcagccaatgagtgccggcggctgggtgcccggctggccaccacgggccagctctacctggcctggcaggctggcatggacatgtgcagcgccggctggctggccgaccgcagcgtgcgctaccccatctccaaggcccggcccaactgcggtggcaacctcctgggcgtgaggaccgtctacgtgcatgccaaccagacgggctaccccgacccctcatcccgctacgacgccatctgctacacaggtgaagactttgtggacatcccagaaaacttctttggagtggggggtgaggaggacatcaccgtccagacagtgacctggcctgacatggagctgccactgcctcgaaacatcactgagggtgaagcccgaggcagcgtgatccttaccgtaaagcccatcttcgaggtctcccccagtcccctggaacccgaggagcccttcacgtttgcccctgaaataggggccactgccttcgctgaggttgagaatgagactggagaggccaccaggccctggggctttcccacacctggcctgggccctgccacggcattcaccagtgaggacctcgtcgtgcaggtgaccgctgtccctgggcagccgcatttgccagggggggtcgtcttccactaccgcccgggacccacccgctactcgctgacctttgaggaggcacagcaggcctgcctgcgcacgggggcggtcattgcctcgccggagcagctccaggccgcctacgaagcaggctatgagcagtgtgacgccggctggctgcgggaccagaccgtcagataccccattgtgagcccccggaccccatgcgtgggtgacaaggacagcagcccaggggtcaggacctatggcgtgcgcccatcaacagagacctacgatgtctactgctttgtagacagacttgagggggaggtgttcttcgccacacgccttgagcagttcaccttccaggaagcactggagttctgtgaatctcacaatgctacgctggccaccacgggccagctctacgccgcctggagccgcggcctggacaagtgctatgccggctggctggccgacggcagcctccgctaccccatcgtcaccccaaggcctgcctgcggtggggacaagccaggcgtgagaacggtctacctctaccctaaccagacgggcctcccagacccactgtcccggcaccatgccttctgcttccgaggcatttcagcggttccttctccaggagaagaagagggtggcacacccacatcaccctctggtgtggaggagtggatcgtgacccaagtggttcctggtgtggctgctgtccccgtagaagaggagacaactgctgtaccctcaggggagactactgccatcctagagttcaccaccgagccagaaaaccagacagaatgggaaccagcctataccccagtgggcacatccccgctgccagggatccttcctacttggcctcccactggcgcagcaacagaggaaagtacagaaggcccttctgcaactgaagtgccctctgcctcagaggaaccatccccctcagaggtgccattcccctcagaggagccatccccctcagaggaaccattcccctcagtgaggccattcccctcagtggagctgttcccctcagaggagccattcccctccaaggagccatccccctcagaggaaccatcagcctcggaagagccgtatacaccttcaccccccgtgcccagctggactgagctgcccagctctggggaggaatctggggcccctgatgtcagtggtgacttcacaggcagtggagatgtttcaggacaccttgacttcagtgggcagctgtcaggggacagggcaagtggactgccctctggagacctggactccagtggtcttacttccacagtgggctcaggcctgcctgtggaaagtggactaccctcaggggatgaagagagaattgagtggcccagcactcctacggttggtgaactgccctctggagctgagatcctagagggctctgcctctggagttggggatctcagtggacttccttctggagaagttctagagacctctgcctctggagtaggagacctcagtgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactactgcccctggagtagaggagatcagcgggcttccttctggagaagttctagagactactgcccctggagtagatgagatcagtgggcttccttctggagaagttctagagactactgcccctggagtagaggagatcagcgggcttccttctggagaagttctagagacttctacctctgcggtaggggacctcagtggacttccttctggaggagaagttctagagatttctgtctctggagtagaggacatcagtgggcttccttctggagaggttgtagagacttctgcctctggaatagaggatgtcagtgaacttccttcaggagaaggtctagagacctctgcttctggagtagaggacctcagcaggctcccttctggagaagaagttctagagatttctgcctctggatttggggacctcagtggacttccttctggaggagaaggtctagagacctctgcttctgaagtagggactgacctcagtgggcttccttctggaagggagggtctagagacttcagcttctggagctgaggacctcagtgggttgccttctggaaaagaagacttggtggggtcagcttctggagacttggacttgggcaaactgccttctggaactctaggaagtgggcaagctccagaaacaagtggtcttccctctggatttagtggtgagtattctggggtggaccttggaagtggcccaccctctggcctgcctgactttagtggacttccatctggattcccaactgtttccctagtggattctacattggtggaagtggtcacagcctccactgcaagtgaactggaagggaggggaaccattggcatcagtggtgcaggagaaatatctggactgccctccagtgagctggacattagtgggagagctagtggactcccttcaggaactgaactcagtggccaagcatctgggtctcctgatgtcagtggggaaatacctggactctttggtgtcagtggacagccatcagggtttcctgacactagtggggaaacatctggagtgactgagcttagcgggctgtcctctggacaaccaggtattagtggagaagcatctggagttctttatggcactagtcaaccctttggcataactgatctgagtggagaaacatctggggtccctgatctcagtgggcagccttcagggttaccagggttcagtggggcaacatcaggagtccctgacctggtttctggtaccacgagtggcagcggtgaatcttctgggattacatttgtggacaccagtttggttgaagtggcccctactacatttaaagaagaagaaggcttagggtctgtggaactcagtggcctcccttccggagaggcagatctgtcaggcaaatctgggatggtggatgtcagtggacagttttctggaacagtcgattccagtgggtttacatcccagactccggaattcagtggcctaccaagtggcatagctgaggtcagtggagaatcctccagagctgagattgggagcagcctgccctcgggagcatattatggcagtggaactccatctagtttccccactgtctctcttgtagacagaactttggtggaatctgtaacccaggctccaacagcccaagaggcaggagaagggccttctggcattttagaactcagtggtgctcattctggagcaccagacatgtctggggagcattctggatttctggacctaagtgggctgcagtccgggctgatagagcccagcggagagccaccaggtactccatattttagtggggattttgccagcaccaccaatgtaagtggagaatcctctgtagccatgggcaccagtggagaggcctcaggacttccagaagttactttaatcacttctgagttcgtggagggtgttactgaaccaactatttctcaggaactaggccaaaggccccctgtgacacacacaccccagctttttgagtccagtggaaaagtctccacagctggggacattagtggagctaccccagtgctccctgggtctggagtagaagtatcatcagtcccagaatctagcagtgagacgtccgcctatcctgaagctgggttcggggcatctgccgcccctgaggccagcagagaagattctgggtcccctgatctgagtgaaaccacctctgcattccacgaagctaaccttgagagatcctctggcctaggagtgagcggcagcactttgacatttcaagaaggcgaggcgtccgctgccccagaagtgagtggagaatccaccaccaccagtgatgtggggacagaggcaccaggcttgccttcagccactcccacggcttctggagacaggactgaaatcagcggagacctgtctggtcacacctcgcagctgggcgttgtcatcagcaccagcatcccagagtctgagtggacccagcagacccagcgccctgcagagacgcatctagaaattgagtcctcaagcctcctgtactcaggagaagagactcacacagtcgaaacagccacctccccaacagatgcttccatcccagcttctccggaatggaaacgtgaatcagaatcaactgctgcagcccccgccaggtcctgtgcagaggagccctgtggagctgggacctgcaaggagacagagggacacgtcatatgcctgtgcccccctggctacactggcgagcactgtaacatagaccaggaggtatgtgaggagggctggaacaagtaccagggccactgttaccgccacttcccggaccgcgagacctgggtggatgctgagcgccggtgtcgggagcagcagtcacacctgagcagcatcgtcacccccgaggagcaggagtttgtcaacaacaatgcccaagactaccagtggatcggcctgaacgacaggaccatcgaaggggacttccgctggtcagatggacaccccatgcaatttgagaactggcgccccaaccagcctgacaacttttttgccgctggagaggactgtgtggtgatgatctggcacgagaagggcgagtggaatgatgttccctgcaattaccacctccccttcacgtgtaaaaagggcacagtggcctgcggagagccccctgtggtggagcatgccaggaccttcgggcagaagaaggaccggtatgagatcaattccctggtgcggtaccagtgcacagaggggtttgtccagcgccacatgcccaccatccggtgccagcccagcgggcactgggaggagcctcagatcacctgcacagaccccaccacctacaaacgcagactacagaagcggagctcacggcaccctcggaggagccgccccagcacagcccactgagaagagcttccaggacgcacccaggacgctgagcccaggagcctgccaggctgacgtgcatcccacccagacggtgtcctcttcttgtcgctttttgtcatataaggaatcccattaaagaaggaaaaaaataaatcccacatttgtgtatgcacccactcacccctccaaatcagcaaaaccgcatctaatttgtccgccgaatgccaaagcaaagcaaacttattataacccttggactgagtttagagacatttcttcaatttcccatcgtgcctttccagggaccagtgcagggacagggggagaaggggaggggttaagttaaataaagaagattatttttgtttcctgactttatccaagagcagtgcaatcgttggttatttcacctccagggagagctagggaggagggaggagggctccaaaggagctggaaggagcagaggcctgagagcaggaagaactcggaaccgcagctgaatgtattggatgagaaggagccaggagggctacaccatctgtatgagggaaaagccttgggggagaggggtgggttcctgcctcctgccgagggtaagccggcaggagagagccatcagagggacctccgctgcctgggagttgggttccctccaagggtccctctttcagtgtcctctctctcacctgggtctgccaccctaacaggtggcaactcggcagggctgctgggggcacttcctgcccagtggggggtgccgcccaaccttctcccctccccacccccgcccccgggaccgtgcaggcaccagggttccgtgcacctatttatatttttgaaaactgaagattataatattataataataataaagacattggaagagat

SEQ ID NO: 3 - DNA, 8960 bp, Homo sapiens
gcccgcccacctacctccccgccgctccagagggggctcgcagagctgaggacgcgcgcagcgctgctcaaggtctctctctctcagcaccctcgccggccggcgtctgacgcgggtgccagggtctccgggcacctttcagtgtccattccctcagccagccaggactccgcaacccagcagttgccgctgcggccacagcccgaggggacctgcggacaggacgccggcaggaggaggggtgcgcagcgcccgcgcagagcgtctccctcgctacgcagcgagacccgggcctcccggccccaggagcccccagctgcctcgccaggtgtgtgggactgaagttcttggagaagggagtccaactcttcaaggtgaactatgaccactttactctgggttttcgtgactctgagggtcatcactgcagctgtcactgtagaaacttcagaccatgacaactcgctgagtgtcagcatcccccaaccgtccccgctgagggtcctcctggggacctccctcaccatcccctgctatttcatcgaccccatgcaccctgtgaccaccgccccttctaccgccccactggccccaagaatcaagtggagccgtgtgtccaaggagaaggaggtagtgctgctggtggccactgaagggcgcgtgcgggtcaacagtgcctatcaggacaaggtctcactgcccaactacccggccatccccagtgacgccaccttggaagtccagagcctgcgctccaatgactctggggtctaccgctgcgaggtgatgcatggcatcgaggacagcgaggccaccctggaagtcgtggtgaaaggcatcgtgttccattacagagccatctctacacgctacaccctcgactttgacagggcgcagcgggcctgcctgcagaacagtgccatcattgccacgcctgagcagctgcaggccgcctacgaagacggcttccaccagtgtgacgccggctggctggctgaccagactgtcagataccccatccacactccccgggaaggctgctatggagacaaggatgagtttcctggtgtgaggacgtatggcatccgagacaccaacgagacctatgatgtgtactgcttcgccgaggagatggagggtgaggtcttttatgcaacatctccagagaagttcaccttccaggaagcagccaatgagtgccggcggctgggtgcccggctggccaccacgggccagctctacctggcctggcaggctggcatggacatgtgcagcgccggctggctggccgaccgcagcgtgcgctaccccatctccaaggcccggcccaactgcggtggcaacctcctgggcgtgaggaccgtctacgtgcatgccaaccagacgggctaccccgacccctcatcccgctacgacgccatctgctacacaggtgaagactttgtggacatcccagaaaacttctttggagtggggggtgaggaggacatcaccgtccagacagtgacctggcctgacatggagctgccactgcctcgaaacatcactgagggtgaagcccgaggcagcgtgatccttaccgtaaagcccatcttcgaggtctcccccagtcccctggaacccgaggagcccttcacgtttgcccctgaaataggggccactgccttcgctgaggttgagaatgagactggagaggccaccaggccctggggctttcccacacctggcctgggccctgccacggcattcaccagtgaggacctcgtcgtgcaggtgaccgctgtccctgggcagccgcatttgccagggggggtcgtcttccactaccgcccgggacccacccgctactcgctgacctttgaggaggcacagcaggcctgcctgcgcacgggggcggtcattgcctcgccggagcagctccaggccgcctacgaagcaggctatgagcagtgtgacgccggctggctgcgggaccagaccgtcagataccccattgtgagcccccggaccccatgcgtgggtgacaaggacagcagcccaggggtcaggacctatggcgtgcgcccatcaacagagacctacgatgtctactgctttgtagacagacttgagggggaggtgttcttcgccacacgccttgagcagttcaccttccaggaagcactggagttctgtgaatctcacaatgctacgctggccaccacgggccagctctacgccgcctggagccgcggcctggacaagtgctatgccggctggctggccgacggcagcctccgctaccccatcgtcaccccaaggcctgcctgcggtggggacaagccaggcgtgagaacggtctacctctaccctaaccagacgggcctcccagacccactgtcccggcaccatgccttctgcttccgaggcatttcagcggttccttctccaggagaagaagagggtggcacacccacatcaccctctggtgtggaggagtggatcgtgacccaagtggttcctggtgtggctgctgtccccgtagaagaggagacaactgctgtaccctcaggggagactactgccatcctagagttcaccaccgagccagaaaaccagacagaatgggaaccagcctataccccagtgggcacatccccgctgccagggatccttcctacttggcctcccactggcgcagcaacagaggaaagtacagaaggcccttctgcaactgaagtgccctctgcctcagaggaaccatccccctcagaggtgccattcccctcagaggagccatccccctcagaggaaccattcccctcagtgaggccattcccctcagtggagctgttcccctcagaggagccattcccctccaaggagccatccccctcagaggaaccatcagcctcggaagagccgtatacaccttcaccccccgtgcccagctggactgagctgcccagctctggggaggaatctggggcccctgatgtcagtggtgacttcacaggcagtggagatgtttcaggacaccttgacttcagtgggcagctgtcaggggacagggcaagtggactgccctctggagacctggactccagtggtcttacttccacagtgggctcaggcctgcctgtggaaagtggactaccctcaggggatgaagagagaattgagtggcccagcactcctacggttggtgaactgccctctggagctgagatcctagagggctctgcctctggagttggggatctcagtggacttccttctggagaagttctagagacctctgcctctggagtaggagacctcagtgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccactgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagaccgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactgctgcccctggagtagaggacatcagcgggcttccttctggagaagttctagagactactgcccctggagtagaggagatcagcgggcttccttctggagaagttctagagactactgcccctggagtagatgagatcagtgggcttccttctggagaagttctagagactactgcccctggagtagaggagatcagcgggcttccttctggagaagttctagagacttctacctctgcggtaggggacctcagtggacttccttctggaggagaagttctagagatttctgtctctggagtagaggacatcagtgggcttccttctggagaggttgtagagacttctgcctctggaatagaggatgtcagtgaacttccttcaggagaaggtctagagacctctgcttctggagtagaggacctcagcaggctcccttctggagaagaagttctagagatttctgcctctggatttggggacctcagtggacttccttctggaggagaaggtctagagacctctgcttctgaagtagggactgacctcagtgggcttccttctggaagggagggtctagagacttcagcttctggagctgaggacctcagtgggttgccttctggaaaagaagacttggtggggtcagcttctggagacttggacttgggcaaactgccttctggaactctaggaagtgggcaagctccagaaacaagtggtcttccctctggatttagtggtgagtattctggggtggaccttggaagtggcccaccctctggcctgcctgactttagtggacttccatctggattcccaactgtttccctagtggattctacattggtggaagtggtcacagcctccactgcaagtgaactggaagggaggggaaccattggcatcagtggtgcaggagaaatatctggactgccctccagtgagctggacattagtgggagagctagtggactcccttcaggaactgaactcagtggccaagcatctgggtctcctgatgtcagtggggaaatacctggactctttggtgtcagtggacagccatcagggtttcctgacactagtggggaaacatctggagtgactgagcttagcgggctgtcctctggacaaccaggtattagtggagaagcatctggagttctttatggcactagtcaaccctttggcataactgatctgagtggagaaacatctggggtccctgatctcagtgggcagccttcagggttaccagggttcagtggggcaacatcaggagtccctgacctggtttctggtaccacgagtggcagcggtgaatcttctgggattacatttgtggacaccagtttggttgaagtggcccctactacatttaaagaagaagaaggcttagggtctgtggaactcagtggcctcccttccggagaggcagatctgtcaggcaaatctgggatggtggatgtcagtggacagttttctggaacagtcgattccagtgggtttacatcccagactccggaattcagtggcctaccaagtggcatagctgaggtcagtggagaatcctccagagctgagattgggagcagcctgccctcgggagcatattatggcagtggaactccatctagtttccccactgtctctcttgtagacagaactttggtggaatctgtaacccaggctccaacagcccaagaggcaggagaagggccttctggcattttagaactcagtggtgctcattctggagcaccagacatgtctggggagcattctggatttctggacctaagtgggctgcagtccgggctgatagagcccagcggagagccaccaggtactccatattttagtggggattttgccagcaccaccaatgtaagtggagaatcctctgtagccatgggcaccagtggagaggcctcaggacttccagaagttactttaatcacttctgagttcgtggagggtgttactgaaccaactatttctcaggaactaggccaaaggccccctgtgacacacacaccccagctttttgagtccagtggaaaagtctccacagctggggacattagtggagctaccccagtgctccctgggtctggagtagaagtatcatcagtcccagaatctagcagtgagacgtccgcctatcctgaagctgggttcggggcatctgccgcccctgaggccagcagagaagattctgggtcccctgatctgagtgaaaccacctctgcattccacgaagctaaccttgagagatcctctggcctaggagtgagcggcagcactttgacatttcaagaaggcgaggcgtccgctgccccagaagtgagtggagaatccaccaccaccagtgatgtggggacagaggcaccaggcttgccttcagccactcccacggcttctggagacaggactgaaatcagcggagacctgtctggtcacacctcgcagctgggcgttgtcatcagcaccagcatcccagagtctgagtggacccagcagacccagcgccctgcagagacgcatctagaaattgagtcctcaagcctcctgtactcaggagaagagactcacacagtcgaaacagccacctccccaacagatgcttccatcccagcttctccggaatggaaacgtgaatcagaatcaactgctgcagcccccgccaggtcctgtgcagaggagccctgtggagctgggacctgcaaggagacagagggacacgtcatatgcctgtgcccccctggctacactggcgagcactgtaacatagacattgatgagtgcctctcaagcccttgtctgaatggagccacctgcgtggatgccatcgactctttcacatgcttatgccttcccagctacgaaggggacctgtgtgagattgaccaggaggtatgtgaggagggctggaacaagtaccagggccactgttaccgccacttcccggaccgcgagacctgggtggatgctgagcgccggtgtcgggagcagcagtcacacctgagcagcatcgtcacccccgaggagcaggagtttgtcaacaacaatgcccaagactaccagtggatcggcctgaacgacaggaccatcgaaggggacttccgctggtcagatggacaccccatgcaatttgagaactggcgccccaaccagcctgacaacttttttgccgctggagaggactgtgtggtgatgatctggcacgagaagggcgagtggaatgatgttccctgcaattaccacctccccttcacgtgtaaaaagggcacagtggcctgcggagagccccctgtggtggagcatgccaggaccttcgggcagaagaaggaccggtatgagatcaattccctggtgcggtaccagtgcacagaggggtttgtccagcgccacatgcccaccatccggtgccagcccagcgggcactgggaggagcctcagatcacctgcacagaccccaccacctacaaacgcagactacagaagcggagctcacggcaccctcggaggagccgccccagcacagcccactgagaagagcttccaggacgcacccaggacgctgagcccaggagcctgccaggctgacgtgcatcccacccagacggtgtcctcttcttgtcgctttttgtcatataaggaatcccattaaagaaggaaaaaaataaatcccacatttgtgtatgcacccactcacccctccaaatcagcaaaaccgcatctaatttgtccgccgaatgccaaagcaaagcaaacttattataacccttggactgagtttagagacatttcttcaatttcccatcgtgcctttccagggaccagtgcagggacagggggagaaggggaggggttaagttaaataaagaagattatttttgtttcctgactttatccaagagcagtgcaatcgttggttatttcacctccagggagagctagggaggagggaggagggctccaaaggagctggaaggagcagaggcctgagagcaggaagaactcggaaccgcagctgaatgtattggatgagaaggagccaggagggctacaccatctgtatgagggaaaagccttgggggagaggggtgggttcctgcctcctgccgagggtaagccggcaggagagagccatcagagggacctccgctgcctgggagttgggttccctccaagggtccctctttcagtgtcctctctctcacctgggtctgccaccctaacaggtggcaactcggcagggctgctgggggcacttcctgcccagtggggggtgccgcccaaccttctcccctccccacccccgcccccgggaccgtgcaggcaccagggttccgtgcacctatttatatttttgaaaactgaagattataatattataataataataaagacattggaagaga

(19) United States
(12) Patent Application Publication
(10) Pub. No.: US 2023/0031371 A1
(43) Pub. Date: Feb. 2, 2023

(54) DIAGNOSIS OF AN AORTIC DISSECTION BY DETECTING A SPECIFIC BIOMARKER IN A BLOOD SAMPLE

(71) Applicant: DEUTSCHES HERZZENTRUM MUENCHEN, Munich (DE)

(72) Inventors: Ruediger LANGE, Munich (DE); Markus KRANE, Munich (DE); Martina DRESSEN, Dachau (DE); Harald LAHM, Munich (DE); Christian KOENIG, Munich (DE)

(21) Appl. No.: 17/791,563

(22) PCT Filed: Dec. 21, 2020
(86) PCT No.: PCT/EP2020/087418

§ 371 Date: Jul. 8, 2022

(30) Foreign Application Priority Data
Jan. 10, 2020 (EP) 20151237.3

(51) Int. Cl. G01N 33/68

(52) U.S. Cl. CPC G01N 33/6887; G01N 33/6854; G01N 33/6893; G01N 2800/329

(57) ABSTRACT

A method of diagnosing an aortic dissection in a sample is disclosed. A kit for diagnosing an aortic dissection in a sample is also disclosed. A point-of-care device is also disclosed for performing the method of diagnosing an aortic dissection

### CROSS-REFERENCE TO RELATED APPLICATION

The present application is a national stage entry according to 35 U.S.C. § 371 of PCT application No.: PCT/EP2020/087418 filed on Dec. 21, 2020; which claims priority to European patent application 20151237.3, filed on Jan. 10, 2020; all of which are incorporated herein by reference in their entirety and for all purposes.

### REFERENCE TO A SEQUENCE LISTING SUBMITTED VIA EFS-WEB

The content of the ASCII text file of the sequence listing named “P85197US_seqlist_ST25”, which is 35 kb in size was created on Jan. 10, 2020. The sequence listing was corrected on Jul. 8, 2022 to correctly delete the information pertaining to the protein isoform precursor; the corrected sequence listing is electronically submitted via EFS-Web herewith; the original sequence listing and the corrected sequence listing are incorporated by reference in their entirety.

### TECHNICAL FIELD

The present disclosure relates to a method of diagnosing an aortic dissection in a sample and further relates to a use of aggrecan or a variant thereof as a biomarker for diagnosing an aortic dissection in a sample. The present disclosure also relates to a kit for diagnosing an aortic dissection in a sample and further relates to a point-of-care device for performing the method of diagnosing an aortic dissection.

### BACKGROUND

An aortic dissection, such as a type A aortic dissection, is a life-threating condition which occurs when an injury to the innermost layer of the aorta, a so called intimal tear, allows blood to flow between the layers of the aortic wall, thereby forcing the layers apart. Presently, the diagnosis is predominantly based on medical imaging, such as computed tomography or ultrasound used to confirm and evaluate a dissection. Aortic dissections are basically classified into two main types, type A and B, after the Stanford classification according to the anatomic region of the intimal tear. In Stanford type A dissections the intimal tear lies ahead of the left subclavian artery and in Stanford B dissections the intimal tear lies beyond the left subclavian artery accordingly.

Fatal rupture of the aorta is the most devastating complication of acute aortic dissections, especially in type A dissections. Therefore, without immediate surgical restoration of the aorta, the mortality rate in patients having a type A aortic dissection is approximately 1-5%/hour. Due to this highly acute and life-threatening characteristic of aortic dissections, particularly type A aortic dissections, an immediate diagnosis and subsequent surgical treatment is necessary.

The clinical symptoms of a patient at the time of admission of the patient play the major role for diagnosing an aortic dissection. Such clinical symptoms may include an acute severe pain in the chest and/or back. An important differential diagnosis of a type A aortic dissection has to be performed, since the clinical symptoms of a heart attack may be similar to the clinical symptoms of an aortic dissection. Presently there are multiple reliable biomarkers that may be used to diagnose a heart attack in a blood sample. However, for an aortic dissection, particularly a type A aortic dissection, such biomarkers are presently missing. Therefore, in patients with acute chest pain, biomarkers for myocardial damage and for a possible aortic dissection should be immediately evaluated as a basic routine. This algorithm will prevent the responsible physician to put the patient on the wrong medication. Anticoagulant drugs, commonly used as treatment in acute coronary syndromes would be fatal in patients with acute aortic dissections. Furthermore the likelihood of missing out the diagnosis of an acute aortic dissection, after acute myocardial infarction was ruled out, will be reduced significantly. Additionally the time between diagnosis and definitive treatment will be considerably reduced. An early indication of the underlying disease process due to suitable biomarkers will prompt the appropriate further diagnostic tests sooner. Therefore, there is a need for suitable biomarkers for aortic dissections, particularly a type A aortic dissection, and for methods of diagnosing aortic dissections.

Many clinics do not have the capability of performing differential diagnosis tests in due time, since high resolution imaging data is required for performing a differential diagnosis of an aortic dissection. Therefore, a specific biomarker is needed which is a first and early indicator of the occurrence of a type A aortic dissection in a patient, so that subsequent respective medical examinations can be performed in a targeted and timely manner.

Due to the unspecific clinical symptoms, aortic dissections are often not diagnosed at all or diagnosed too late. Presently, the diagnosis of a type A aortic dissection is based on complex, labor-intensive, and cost-intensive medical examinations, such as computed tomography involving contrast agents. Therefore, a method for diagnosing type A aortic dissection involving only simple and fast techniques, such as the detection in a blood sample, would be highly desirable.

Cikach et al. [1] have detected aggrecan in an aorta sample of a patient having an aortic dissection. US 2005/0124071 A1 relates to aggrecan as a biomarker for diseases such as arthritis and joint diseases. EP 2019318 A1 relates to aggrecan as a biomarker for analyzing plaque samples of patients possibly suffering from cardiovascular diseases, such as an abdominal aortic aneurysm. However, a biomarker for detecting an aortic dissection in a blood sample of a patient is currently missing.

Thus, it is an aim to provide a method for diagnosing an aortic dissection, preferably a type A aortic dissection, using a biomarker that is detectable in blood of patients suffering from a type A aortic dissection. Furthermore, it is an aim to provide a method for diagnosing an aortic dissection, preferably a type A aortic dissection in an easy, reliable, and timely manner.

### SUMMARY

In the following, the elements will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, a method of diagnosing an aortic dissection in a sample may include:
a) providing a sample of a patient, wherein said sample is a blood sample, a serum sample, and/or a plasma sample,
b) measuring a level of aggrecan or a variant thereof, optionally measuring a level of at least one further biomarker other than aggrecan,
c) comparing the level(s) measured to a respective reference value and/or a reference sample of a healthy person not suffering from an aortic dissection.

In one embodiment, said method further comprises a step of determining that a patient has an aortic dissection, if the level of aggrecan, optionally the level of said further biomarker other than aggrecan, in said sample is increased compared to the reference value and/or reference sample.

In one embodiment, said aortic dissection is selected from a type A aortic dissection and a type B aortic dissection, preferably is a type A aortic dissection.

In one embodiment, said sample is a human sample.

In one embodiment, said biomarker other than aggrecan is selected from OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23, TAGLN2, TAGLN3, THBS2, VCAN, and variants thereof, preferably is selected from OGN, LPYD, ITGA11, and variants thereof.

In one embodiment, said measuring is performed by a method selected from ELISA, PCR, qPCR, flow cytometry, mass spectrometry, antibody-based protein chips, 2-dimensional gel electrophoresis, western blot, protein immuno-precipitation, radio immunoassay, ligand binding assay, and liquid chromatography, preferably selected from ELISA; radio immunoassay, and antibody-based protein chips.

In one embodiment, said level refers to a level of protein and/or nucleic acid.

A method of diagnosing an aortic dissection in a sample may include:
i) measuring a level of aggrecan or a variant thereof in a sample of a patient, wherein said sample is a blood sample, a serum sample, and/or a plasma sample, optionally measuring a level of at least one further biomarker other than aggrecan in said sample,
ii) comparing the level(s) measured to a respective reference value and/or a reference sample of a healthy person not suffering from an aortic dissection.

In one embodiment, said method further comprises a step of determining that said patient has an aortic dissection, if the level of aggrecan, optionally the level of said further biomarker other than aggrecan, in said sample is increased compared to the reference value and/or reference sample.

In one embodiment, said sample of a patient is a sample obtained from a patient. In one embodiment, said aortic dissection, said sample, said biomarker, said measuring, and said level are as defined above.

In a further aspect, aggrecan or a variant thereof as a biomarker may be used for diagnosing an aortic dissection in a sample, wherein said sample is a blood sample, a serum sample and/or a plasma sample.

In one embodiment, said aortic dissection is selected from a type A aortic dissection and a type B aortic dissection, preferably is a type A aortic dissection.

In one embodiment, said sample is a human sample.

In one embodiment, said use further comprises at least one biomarker other than aggrecan for diagnosing an aortic dissection in a sample, said biomarker other than aggrecan preferably being selected from OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23, TAGLN2, TAGLN3, THBS2, VCAN, and variants thereof, more preferably being selected from OGN, LPYD, ITGA11, and variants thereof.

In one embodiment, said biomarker is present in said sample in the form of a protein and/or a nucleic acid, or a fragment thereof.

In this aspect, said aggrecan, said variant, said biomarker, said diagnosing, said aortic dissection, and said sample are as defined above.

In a further aspect, kit for diagnosing an aortic dissection in a sample may include
an agent or means for detecting aggrecan as a biomarker, preferably an antibody or an antigen-binding peptide,
optionally a reference means, preferably a reference sample of a healthy person or a recombinant aggrecan in a defined amount,
optionally one or more agent(s) or means for detecting a biomarker other than aggrecan,
optionally auxiliary compounds for performing the method as defined in any of the embodiments above,
optionally comprising instructions for diagnosing an aortic dissection, particularly for comparing the aggrecan level measured, optionally the level of at least one further biomarker measured, to a reference value and/or a reference sample of a healthy person not suffering from an aortic dissection, wherein an increase in the aggrecan level, optionally the level of a further biomarker, indicates an aortic dissection.

In this aspect, said aggrecan, said biomarker, said diagnosing, said aortic dissection, said measured, and said sample are as defined above.

In a further aspect, a point-of-care device, for performing the method of diagnosing an aortic dissection as defined in any of the embodiments above, may include:
a sample inlet for contacting a sample selected from a blood sample, a serum sample and/or a plasma sample with said point-of-care device,
an analyzing unit for measuring an aggrecan level in said sample, optionally further measuring at least one biomarker other than aggrecan in said sample,
an evaluation unit comprising a detector for detecting an aggrecan level, wherein said detector generates an output signal indicating an aggrecan level.

In this aspect, said method, said diagnosing, said aortic dissection, said sample, said aggrecan, and said biomarker are as defined above.

In a further aspect, the present disclosure also relates to a method of treatment of an aortic dissection, preferably a type A aortic dissection, comprising diagnosing said aortic dissection, preferably said type A aortic dissection using a method of diagnosing an aortic dissection as defined above.

In this aspect, said aortic dissection and said method of diagnosing are as defined above.

In a further aspect, at least one biomarker may be used for the manufacture of a medicament for the diagnosis and/or treatment of an aortic dissection, preferably a type A aortic dissection, wherein said at least one biomarker is aggrecan and optionally any selected from OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23, TAGLN2, TAGLN3, THBS2, and VCAN.

In this aspect, said biomarker, said diagnosis, and said aortic dissection are as defined above.

In a further aspect, aggrecan, and optionally additionally any biomarker other than aggrecan selected from OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23, TAGLN2, TAGLN3, THBS2, and VCAN may be used for diagnosing an aortic dissection, preferably a type A aortic dissection.

In this aspect, said biomarker, said diagnosis, and said aortic dissection are as defined above.

### BRIEF DESCRIPTION OF THE DRAWINGS

The figures will now be described.

All methods mentioned in the figure descriptions below were carried out as described in detail in the examples.

FIG. 1 shows gene expression in different surgical biopsies. Skel: skeletal muscle (n=5), fat: subcutaneous fat (n=5), LA: left atrium (n=5), AoT: aorta from type A dissection (n=6), AoC: aorta from coronary artery bypass graft (n=6), V: vena saphena magna (n=5), IMA: arteria mammaria interna (n=5). Depicted are RNA expression data of the biomarker candidates hyaluronan and proteoglycan link protein 1 (HAPLN1), integrin alpha-11 (ITGA11), osteoglycin (OGN), and aggrecan (ACAN). Values represent means±SEM. Significance of differences was tested with one-way ANOVA test.

FIG. 2 shows plasma concentrations of the four biomarker candidates ACAN, ITGA11, LPYD, and OGN; Type A dissections (ao type A, group 1, n=14), healthy control group (ctrl, group 5, n=7), mitral valve insufficiency (MKP, group 2, n=10). Values represent means±SEM. Significance of differences was tested with one-way ANOVA test.

FIG. 3 depicts plasma concentrations of biomarker candidate ACAN; type A dissections (ao type A, group 1, n=11 of experiment 1, n=4 new samples), healthy control group (ctrl, group 5, n=5), Aneurysma verum (true aneurysm, group 3, n=7), and surgical cryoablation (MAZE, group 4, n=5). Significance of differences was tested with one-way ANOVA test.

FIG. 4 shows the fold change of plasma concentrations of the biomarker candidate ACAN in individual samples compared to the mean concentration of the control group (group 5). Depicted are the respective duplicate measurements for each patient from two independent assays.

FIG. 5 depicts the fold change of plasma concentrations of the biomarker candidate ACAN compared to the mean concentration of the control group (group 5). Depicted are the mean □ SEM values of two independent experiments. Assay 1: ctrl n=7, ao type A n=14, Assay 2: ctrl n=5, ao type A n=15. Results represent the mean±SEM. Significance of differences was tested with unpaired two-tailed Student's t test (assay 2) or the Mann-Whitney Rank Sum test if the equal variance or normality test failed (assay 1).

FIG. 6 shows an exemplary workflow for identifying candidate markers for acute type A aortic dissection patients and expression of selected candidate genes in human heart regions and surgical biopsies. A) Strategy for selection and measurement of candidate biomarkers. B) Protein expression of ACAN in different regions of the human heart. Ao: aorta, AV: aortic valve, RCA: right coronary artery, LA: left atrium, LCA: left coronary artery, LV: left ventricle, MV: mitral valve, PA: pulmonary artery, PV: pulmonary valve, Pve: pulmonary vein, RA: right atrium, RV: right ventricle, SepA: atrial septum, SepV: ventricular septum, TV: tricuspid valve, IVC: inferior vena cava.

FIG. 7 shows that ACAN levels are not enhanced in plasma samples of patients without acute type A aortic dissection (ATAAD). ACAN concentration in plasma of patients with ATAAD (type A, n=33), asymptomatic chronic aneurysm of the ascending aorta (aneurysm, n=13), myocardial infarction (MI) with an acute ST-elevation (STEMI, n=18), without known coronary artery disease (N-CAD, n=15) and healthy volunteers (control, n=12). Values represent means±SEM. Significance of differences was tested with one-way ANOVA test.

FIG. 8 shows that ACAN levels in ATAAD patients are not influenced by basic demographic parameters or the course of ATAAD. A) ACAN levels in female (n=15) and male (n=18) ATAAD patients. B) ACAN levels in ATAAD patients at the age of 40-49 years (n=3), 50-59 years (n=6), 60-69 years (n=12), 70-79 years (n=9) and 80 years or older (n=3). C) ACAN levels in ATAAD patients with DeBakey type I (n=14) or type II (n=12). D) ACAN levels at 6 (n=8), 12 (n=8), 24 (n=6), 48 (n=3) or 72 h (n=2) after the onset of ATAAD. Values are presented as means±SEM. Significance of differences was tested with Wilcoxon-Mann-Whitney test (A and C) or one-way ANOVA test followed by Dunn's or Holm-Sidak Method (B and D).

FIG. 9 shows that ACAN levels do not correlate with CK-MB or cTnT concentrations. A) CK-MB levels of individual ATAAD patients. B) Correlation between ACAN and CK-MB levels. C) cTnT levels of individual ATAAD patients. D) Correlation between ACAN and cTnT levels. ACAN: aggrecan, CK-MB: creatine kinase-muscle brain isoform, cTnT: cardiac troponin T, STEMI: ST-elevation-myocardial-infarction.

FIG. 10 shows sensitivity and specificity of ACAN to detect ATAAD. A) Receiver-operating characteristics curve for all patients with ATAAD (n=33) vs. all control subjects (n=63). B) ACAN levels in ATAAD patients (n=33). C) ACAN levels in STEMI patients (n=18). D) ACAN levels in patients with aneurysm (n=13). E) ACAN levels in surgical controls (MVR) (●, n=9), controls without coronary artery disease (N-CAD) (♦, n=15) and healthy persons (▪, n=12). The dashed line refers to the optimum discrimination level of 14.3 ng/mL determined by the ROC analysis. Wrongly grouped samples are indicated in grey. ACAN: Aggrecan, ATAAD: acute thoracic aortic dissection, MVR: mitral valve repair, N-CAD: no coronary artery disease, ROC: receiver-operator-curve, STEMI: ST-elevation-myocardial-infarction.

### DETAILED DESCRIPTION

A non-limiting embodiment allows for diagnosing an aortic dissection, particularly a type A aortic dissection, in a blood sample of a patient. The present inventors herein disclose aggrecan to be a suitable biomarker for use in a method of diagnosing an aortic dissection, preferably a type A aortic dissection. Particularly, the present inventors detected that aggrecan levels are increased in blood samples of patients suffering from a type A dissection compared to subjects not suffering from a type A dissection.

The method unexpectedly allows for diagnosing an acute type A aortic dissection in a blood sample. Particularly, an advantage of a method is that provides a biomarker, such as aggrecan, which can be analyzed quickly, easily, and noninvasively. In case aggrecan is detected as being increased in the plasma or serum of a patient, the patient can be promptly directed to further medical examinations, such as computed tomography, and/or an immediate surgery can be performed. A further advantage of a method of diagnosing an aortic dissection using aggrecan as a biomarker is that it allows for an easy and fast diagnosis in an emergency room or at any location of first aid, for example by using a kit and/or a point-of-care device provides a fast and reliable diagnosis of an aortic dissection, preferably as fast as <3.5 h, more preferably as fast as <1 h.

The term “diagnosing” or “diagnosis”, as used herein, relates to inspecting a patient's health condition, preferably relates to determining which disease or condition causes a person's symptoms and/or health condition. In one embodiment, diagnosing relates to determining whether a person has an aortic dissection, such as a type A aortic dissection. In one embodiment, a diagnosis comprises a differential diagnosis, in which it is determined whether a patient has a heart attack, an aortic dissection, or neither of the two. In one embodiment, a method of diagnosing comprises using aggrecan and any biomarker selected from OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23, TAGLN2, TAGLN3, THBS2, and VCAN for determining whether a subject has an aortic dissection, and optionally further comprises using any biomarker selected from troponin I, troponin T, hs troponin, and CK-MB for determining whether a subject has a heart attack. In one embodiment, the method of diagnosing an aortic dissection in a sample is performed extracorporally; in particular, the step (b) of measuring a level of aggrecan or a variant thereof, optionally measuring a level of at least one further biomarker, is performed extracorporally. In one embodiment, the method of diagnosing an aortic dissection is performed extracorporally using a blood sample obtained from a patient.

In one embodiment, if a method of diagnosing has indicated that a patient has an aortic dissection, said method of diagnosing further comprises a subsequent step of imaging said patient, such as by computed tomography or ultrasound, prior to performing a surgical treatment.

The term “aortic dissection”, as used herein, refers to a serious medical condition in which the inner layer of the aorta tears. Blood may surge through the tear which causes the inner and middle layers of the aorta to dissect. Aortic dissection can be fatal due to not enough blood flowing to the heart or complete rupture of the aorta. The Stanford classification describes two types of aortic dissections, namely type A and type B aortic dissections, depending on whether the ascending aorta is involved. Type A involves the ascending aorta and/or aortic arch, and in some cases the descending aorta. Type B involves the descending aorta or the arch (distal to the left subclavian artery), without involving the ascending aorta. Type A ascending aortic dissections generally require primary surgical treatment, whereas type B dissections generally are treated medically as an initial treatment and surgery or intervention is used if any complications occur.

The term “type A aortic dissection”, as used herein, refers to an aortic dissection in which a tear occurs typically in the ascending part of the aorta. A type A aortic dissection may also affect any of the aortic root, aortic arch, and the entire aorta. Type A dissections may result from hypertension, inherited connective tissue weakness and/or an aneurysm. Subjects with an acute type A aortic dissection typically experience a sudden onset of severe chest pain, which may spread to the neck, jaw or back. Symptoms such as shortness of breath, loss of consciousness, and symptoms similar to those from a stroke, such as sudden difficulty speaking, visual loss, and weakness may occur. Type A aortic dissection urgently requires diagnosis and surgery. Without treatment, about 50% of patients with Stanford type A dissections die within three days. In one embodiment, cystic medial degeneration, which plays a role in type A aortic dissection, leads to a degeneration of smooth muscle cells, resulting in the production of replacement tissue and/or proteins such as aggrecan.

The term “sample”, as used herein, refers to a sample of a patient, preferably a blood sample. In one embodiment, said sample is a blood sample, a serum sample and/or a plasma sample. In one embodiment, the term “blood sample” relates to a whole blood sample, a serum sample, and/or plasma sample. In one embodiment, said patient is a human. An advantage is that a method of diagnosing can be performed using only very small amounts of blood, such as 10-1000 μl, preferably <500 μl of blood, more preferably ≤100 μl of blood. In one embodiment, the term “sample of a patient” refers to a sample obtained from a patient, preferably a blood sample. In one embodiment, a sample of a patient is obtained by minimally invasive and/or non-invasive blood drawing, preferably non-invasive blood drawing. In one embodiment, providing a sample of a patient and/or obtaining a sample from a patient involve(s) merely minimally invasive and/or non-invasive techniques, preferably non-invasive techniques. In one embodiment, providing a sample of a patient and/or obtaining a sample from a patient involve(s) merely uncritical methods involving only a minor intervention and no substantial health risks for said patient.

The term “measuring”, as used herein, refers to quantifying a parameter in a sample, particularly to quantifying a level of a biomarker such as aggrecan. In one embodiment, measuring a biomarker level is performed using any method, such as any of ELISA, PCR, qPCR, flow cytometry, mass spectrometry, antibody-based protein chips, 2-dimensional gel electrophoresis, western blot, protein immuno-precipitation, radio immunoassay, ligand binding assay, and liquid chromatography, preferably selected from ELISA; radio immunoassay, and antibody-based protein chips. In one embodiment, aggrecan and optionally one or more other biomarker(s) are measured. In one embodiment, the levels of aggrecan and any of osteoglycin, lysyl pyridinoline, and integrin alpha 11 are measured.

The term “level”, as used herein, refers to a level of protein and/or nucleic acid of a biomarker in a sample. In one embodiment, the level of a biomarker is an indicator of the biomarker's concentration and/or amount in a sample. In one embodiment, when referring to the presence of a biomarker in a sample, the terms biomarker “level”, “concentration”, and “amount” may be used interchangeably. In one embodiment, the level is measured using an ELISA assay.

The term “aggrecan”, as used herein, refers to a proteoglycan and a component of the extracellular matrix. Aggrecan is predominantly present in hyaline cartilage. Aggrecan (ACAN) is also known as cartilage-specific proteoglycan core protein (CSPCP) or chondroitin sulfate proteoglycan 1, and is a protein that is encoded by the ACAN gene in humans. The human form of the aggrecan protein can be expressed in multiple isoforms due to alternative splicing, particularly isoforms 1 (SEQ ID No. 1; NM_001135.3 aggrecan core protein isoform 1 precursor), 2 (SEQ ID No. 2; NM_013227.3 aggrecan core protein isoform 2 precursor), and 3 (SEQ ID No. 3; NM_001369268.1 aggrecan core protein isoform 3 precursor). The amino acid sequences of isoforms 1, 2, and 3 comprise 2431 aa, 2530 aa, and 2568 aa, respectively. Mutations in the ACAN gene can result in the rare disease Spondyloepiphyseal dysplasia. Aggrecan depositions within the vascular system may result in increased vascular stiffness. In one embodiment, the term “aggrecan” further comprises a biologically active fragment of aggrecan, as well as variants thereof. In one embodiment, aggrecan is used as a biomarker for diagnosing an aortic dissection, preferably a type A aortic dissection.

The term “variant”, as used herein, refers to any derivative of a biomarker, such as a fragment, an isoform, a fragment of an isoform, an alternatively spliced variant, a mutated variant, a posttranslationally modified variant, or fragments thereof. In one embodiment, the variant relates to a variant of a biomarker in the form of a nucleic acid and/or protein. In one embodiment, a variant of a biomarker is a biologically active derivative, such as a biologically active fragment, of said biomarker. In one embodiment, a variant of aggrecan is any of an aggrecan fragment, an isoform of aggrecan (SEQ ID. 1-3) or fragment thereof, an alternatively spliced variant, a mutated variant, and a posttranslationally modified variant. In one embodiment, a variant of a biomarker other than aggrecan is any fragment, isoform, fragment of an isoform, alternatively spliced variant, mutant variant, posttranslationally modified variant of said biomarker.

The term “biomarker”, as used herein, refers to a naturally occurring molecule, gene, protein, and/or characteristic by which a particular pathological or physiological process and/or disease can be identified. In one embodiment, a biomarker is used to diagnose the presence of an aortic dissection, preferably, and type A aortic dissection. In one embodiment, a biomarker used to diagnose an aortic dissection in accordance with a method is any biomarker selected from aggrecan (ACAN), osteoglycin (OGN), lysyl pyridinoline (LPYD), integrin subunit alpha 11 (ITGA11), anoctamin (ANO1), BROX (BRO1 domain and CAAX motif containing), leucine rich melanocyte differentiation associated (LRMDA/C10orf11), CD47 molecule (CD47), calponin 1 (CNN1), cartilage oligomeric matrix protein (COMP), fibulin 2 (FBLN2), fibulin 5 (FBLN5), fibromodulin (FMOD), fibronectin type III domain containing 1 (FNDC1), GULP PTB domain containing engulfment adaptor 1 (GULP1), hyaluronan and proteoglycan link protein 1 (HAPLN1), hyaluronan and proteoglycan link protein 2 (HAPLN2), lysyl oxidase like 1 (LOXL1), latent transforming growth factor beta binding protein 4 (LTBP4), murine retrovirus integration site 1 homolog (MRVI1), myosin heavy chain 9 (MYH9), nephronectin (NPNT), neuronal pentraxin receptor (NPTXR), synaptosome associated protein 23 (SNAP23), transgelin 2 (TAGLN2), transgelin 3 (TAGLN3), thrombospondin 2 (THBS2), versican (VCAN), and variants thereof. In one embodiment, a method of diagnosing comprises measuring a level of the biomarker aggrecan, and optionally additionally measuring a level of at least one other biomarker than aggrecan. In one embodiment, the level of biomarker aggrecan is increased in a blood sample of a patient having a type A aortic dissection compared to the level of aggrecan in a blood sample of a healthy individual. In one embodiment, the levels of aggrecan and of at least one other biomarker selected from OGN, LPYD, and ITGA11 are increased in a blood sample of a patient having a type A aortic dissection compared to the levels in a healthy individual. In one embodiment, the levels of aggrecan and of at least one other biomarker selected from OGN, HAPLN1, and ITGA11 are increased in a blood sample of a patient having a type A aortic dissection compared to the levels in a healthy individual and/or the levels in a patient suffering from a disease other than type A aortic dissection, for example a cardiovascular disease such as a myocardial infarction.

The term “osteoglycin”, as used herein, refers to a human protein which is encoded by the OGN gene. The OGN gene encodes a protein which induces ectopic bone formation in conjunction with transforming growth factor beta. The osteoglycin protein is a small proteoglycan which contains tandem leucine-rich repeats (LRR). The expression level of OGN has been correlated with enlarged hearts and more specifically left ventricular hypertrophy.

The terms “lysyl pyridinoline” and “LPYD”, as used herein, relate to cross-links that are formed from two hydroxylysine residues and a lysine residue, typically in collagen.

The terms “reference”, “reference value” or “reference sample”, as used herein, refer to a control value and/or control sample which indicates the normal level of a biomarker level. In one embodiment, a reference value and a reference sample are a value detected in a healthy individual and a sample obtained from a healthy individual, respectively. In one embodiment, a reference value and/or reference sample is an indicator of an expected value identified in a healthy individual for a biomarker level of interest. In one embodiment, a reference value and/or reference sample allows to compare a biomarker level detected in a patient with a biomarker level expected for and/or detected in a healthy individual. In one embodiment, a reference sample is used to obtain a reference value. In one embodiment, a patient's aggrecan level is compared to a reference value of an aggrecan level, and an increased aggrecan level in said patient compared to said reference value indicates that said patient has an aortic dissection, particularly a type A aortic dissection. In one embodiment, a patient's biomarker level is compared to a reference value of said biomarker level, and a deviating biomarker level, preferably an increased biomarker level, in said patient compared to said reference value indicates that said patient has an aortic dissection, particularly a type A aortic dissection. In one embodiment, a reference derives from a healthy person not suffering from an aortic dissection. In one embodiment, the terms “reference” and “reference value and/or reference sample” are used interchangeably. In one embodiment, the term “comprising” may relate to “consisting of”.

The term “patient”, as used herein, relates to a subject, preferably a human subject, which has an aortic dissection, is at risk of acquiring an aortic dissection, and/or is suspected of having an aortic dissection. The term “healthy person”, as used herein, refers to a person not suffering from an aortic dissection, preferably not suffering from a cardiovascular disease, more preferably not suffering from any disease at all.

The term “kit”, as used herein, refers to relates to a set of reagents that are necessary to perform a method of diagnosing an aortic dissection in a sample. In one embodiment, a kit is a kit that comprises all the components (other than the blood sample) necessary for carrying out a method, such as for carrying out the method using an ELISA, a radio immunoassay, or an antibody-based protein chips as a measurement method. In one embodiment, a kit can be used with a point-of-care device to analyze a blood sample of a patient in a method of diagnosing an aortic dissection.

The term “agent or means for detecting”, as used herein, refers to any agent or means suitable to detect a biomarker, such as aggrecan and/or a biomarker other than aggrecan. In one embodiment, the protein level of said biomarker is detected using an agent or means being any of an antibody, an antigen-binding peptide such as a Fab fragment, a scFv fragment, a diabody, and a Fab2 fragment, or an aptamer. In one embodiment, the nucleic acid level of said biomarker is detected using an agent or means being any of a probe and a primer.

The term “point-of-care device”, as used herein, refers to a device for medical diagnostic testing at or near the place of patient care. In one embodiment, a point-of-care device is used to analyze a blood sample of a patient. In one embodiment, a point-of-care device is used to analyze a blood sample, such as a whole blood sample, a serum sample, or a plasma sample. In one embodiment, the volume of blood analyzed in a single measurement performed with a point-of-care device is less than 1 mL, preferably less than 500 μl of blood, more preferably less than 100 μl of blood. In one embodiment, the result of an analysis performed with a point-of-care device is available as soon as within 5 hours, preferably within 3.5 hours, more preferably within 60 min after the start of the analysis. In one embodiment, a point-of-care device is easy to handle as well as portable, and may be used within a mobile intensive care unit.

The term “sample inlet”, as used herein, relates to an inlet for contacting a sample with a point-of-care (POC) device for performing the method of diagnosing an aortic dissection. In one embodiment, a sample inlet uses capillary force to contact a sample of a patient suspected of having an aortic dissection with a POC device. In one embodiment, the sample inlet receives the blood sample by means of any of injection, dipping, absorption, pressure, underinflation, vacuum, and/or capillary forces. In one embodiment, the sample inlet receives and transfers a blood sample of a patient suspected of having an aortic dissection to the analyzing unit of the POC device.

The term “analyzing unit”, as used herein, refers to a unit capable of analyzing a sample by measuring a level of a biomarker such as aggrecan.

The term “evaluation unit”, as used herein, refers to a unit that comprises a detector capable of detecting a result of a biomarker analysis performed with an analyzing unit. In one embodiment, an evaluation unit detects an aggrecan level, optionally further detects a level of a biomarker other than aggrecan, and gives the result of said detection(s) as an output signal. In one embodiment, an analyzing unit and evaluation unit may be comprised in one unit and/or be one unit capable of performing both functions.

The term “detector”, as used herein, refers to a module that is capable of detecting the result of an analysis of a biomarker level performed with any of ELISA, PCR, qPCR, flow cytometry, mass spectrometry, antibody-based protein chips, 2-dimensional gel electrophoresis, western blot, protein immuno-precipitation, radio immunoassay, ligand binding assay, and liquid chromatography. In one embodiment, a detector generates an output signal indicating a biomarker level.

The term “output signal”, as used herein, refers to a signal which indicates the result of an analysis of an aggrecan level and/or of a level of a biomarker other than aggrecan.

In the following, reference is made to the examples, which are given to illustrate, not to limit the present invention.

### EXAMPLES

### Example 1: Analysis of Gene Expression in Tissue Biopsies by qRT-PCR

Tissue samples were obtained during the operation and were snap-frozen immediately in liquid nitrogen. They were kept at −196° C. until further use. RNA was extracted using the RNeasy Plus Universal kit (Qiagen, Hilden, Germany) according to the manufacturer's recommendation. cDNA was synthesized from 100 ng total RNA using M-MLV reverse transcriptase (100 U), 250 ng random hexamer primers, 10 mM DTT, dNTPs (0.5 mM each), 15 mM MgCl2, 375 mM KCl and 250 mM Tris-HCl pH 8.3 in a final volume of 30 μL. qRT-PCR analyses were performed on a Quant Studio 3 (ThermoFisher, Germering, Germany) using the following conditions: 95° C. for 10 min, 40 cycles of 95° C. for 15 sec and 60° C. for 1 min using 0.3 μM of each primer. The expression of ACTB (□-actin) was used to normalize the expression levels in individual samples. For all tissue types 5 biopsies were analyzed except for ao type A (n=6) and ao CABG (n=9). Tissue samples of the following sites were analyzed:
left atrium (LA)
skeletal muscle (skel. muscle)
subcutaneous fat tissue (fat)
Vena saphena magna (vein)
Arteria thoracica interna (IMA)
aorta (type A dissection) (ao type A)
aorta (coronary artery bypass graft, CABG) (ao CABG/AoC)

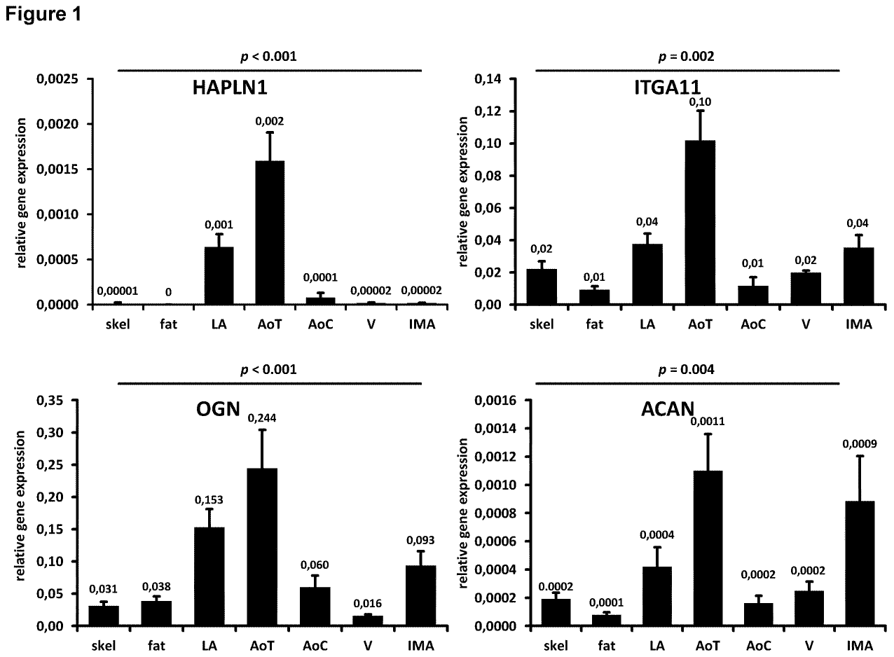

Gene expression of the biomarker candidates detected in the proteomic study was analyzed. FIG. 1 shows the RNA expression data of the biomarker candidates osteoglycin (OGN), hyaluronan and proteoglycan link protein 1 (HAPLN1), integrin alpha-11 (ITGA11) and aggrecan (ACAN). These four biomarker candidates showed increased expression in aorta tissue (ao type A) deriving from a patient having type A dissection compared to the other tissue types. Beta-actin was used as a control housekeeping gene. GOI=Gene of interest.

The biomarker candidates of the 25 biomarker candidates detected in the proteomic study other than OGN, HAPLN1, ITGA11, and ACAN did not show aorta-specific RNA expression.

### Example 2: Evaluation of Biomarkers in Blood Samples

It was analyzed whether it is possible to detect the four identified biomarker candidates, namely OGN, LPYD, ITGA11, and ACAN, or fragments or metabolic products thereof, in a blood sample (such as blood plasma or serum) of a patient having a type A aortic dissection. Pre-surgically obtained plasma samples of various subject populations were analyzed. Particularly, ELISA experiments were performed with samples from four patient groups and one control group.

Aggrecan concentrations in plasma samples were determined using an Enzyme-linked Immunosorbent Assay Kit (Cloud-Clone Corporation, Katy, Tex.) according to the manufacturer's instructions as follows. ACAN standard or plasma (100 μl each) were pipetted into each well and incubated for 1 h at 37° C. The supernatant was aspirated and 100 Detection Reagent A were added and samples were incubated for 1 h at 37° C. Samples were washed three times with 300 μL wash solution, 100 μL Detection Reagent B were added and samples were incubated for 30 min at 37° C. in the dark. Samples were washed three times with 300 μL wash solution and 90 μL TMB substrate solution were added. Samples were incubated for 10 to 20 min at 37° C. in the dark. Thereafter, 50 μL Stop solution were added to each well and the OD 450 nm was determined in an ELISA reader.

With regard to group 3 and 4, only aggrecan was determined. Experiment 1 and experiment 2 were performed following the same experimental protocol except for the samples used.

Group 1: patients having an acute type A aortic dissection

An acute type A aortic dissection was diagnosed for this group of patients by means of computed tomography. Experiment 1: n=14; experiment 2: n=15.

Group 2: patients having an isolated mitral valve insufficiency

Patients of this group suffered from isolated mitral valve insufficiency without having other severe diseases. Pre-surgically obtained samples of this group were analyzed. This group served as a control group. Experiment 1: n=10.

Group 3: patients having an Aneurysma verum of the main artery

Patients of this group suffered from Aneurysma verum of the aorta without having an aortic dissection. Patients of this group did not have an acute, tissue traumatic rupture of the aorta. Presurgically obtained samples of this group were analyzed. This group served as a control group. Experiment 1: n=7.

Group 4: patients having a surgical cryoablation of the atrium in atrial fibrillation Patients of this group (experiment 2: n=5) suffered from a rhythm disorder in the form of atrial fibrillation, which was treated by surgical cryoablation in which a substantial area of the atrial myocardium is selectively destroyed. Postsurgically obtained samples of this group, i.e. samples obtained at the time of admission to the intensive care unit, were analyzed. This group served as a control group.

Group 5: healthy subjects

This control group consists of healthy subjects without a cardiovascular disease. Experiment 1: n=7; experiment 2: n=5.

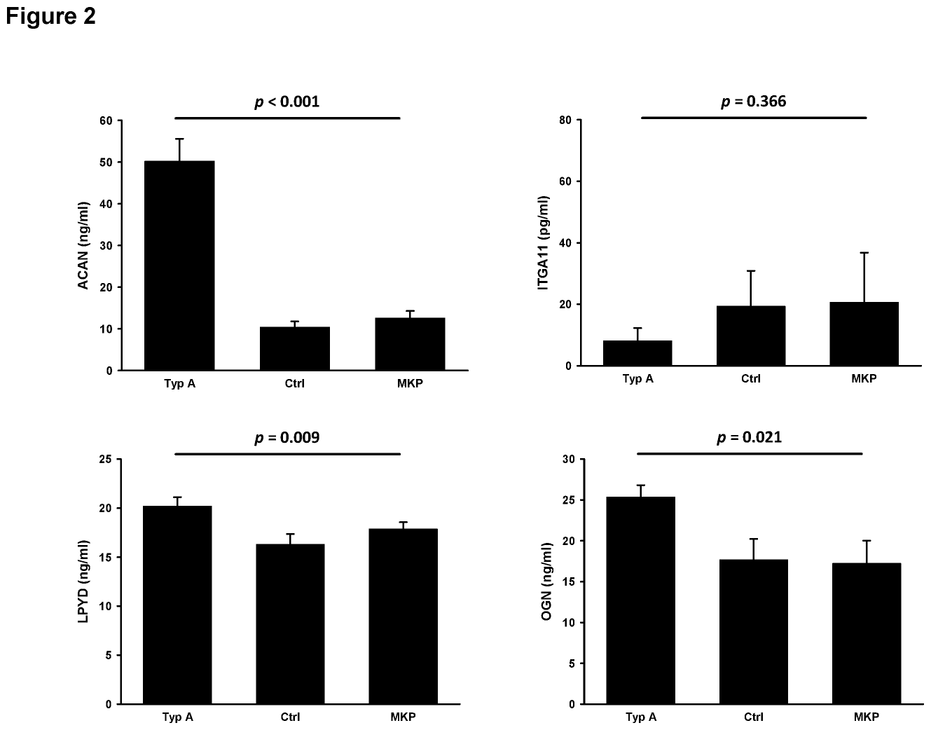

Blood plasma levels of the four most promising biomarker candidates, namely ACAN, OGN, LPYD, and ITGA11, were measured. FIG. 2 shows the measured plasma levels of the respective biomarker candidates in blood plasma of group 1, group 2, and group 5. A significantly increased concentration of ACAN was detected specifically in group 1 using ELISA. OGN and LPYD also showed significantly increased concentrations in group 1. No significant differences were detected with regard to ITGA11.

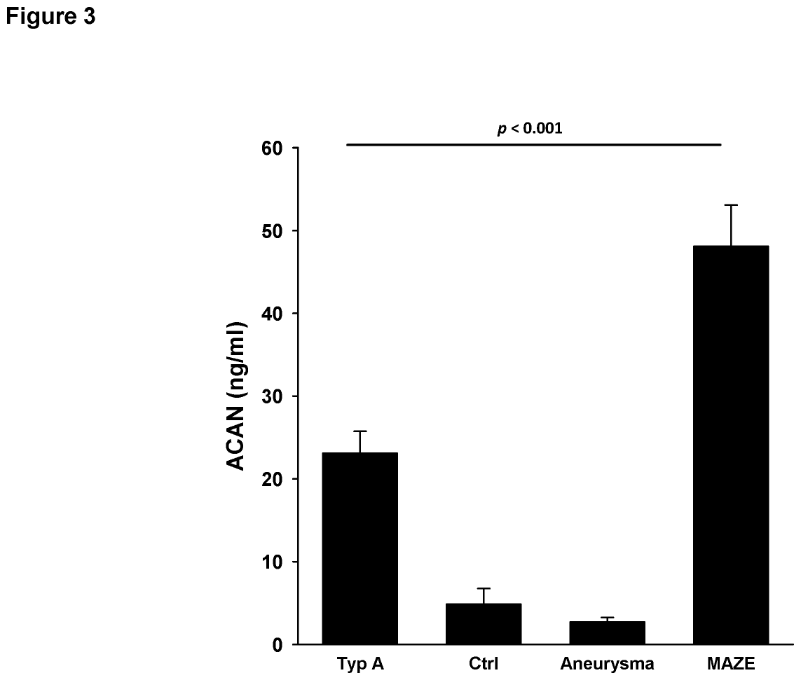

Furthermore, groups 3 and 4 were analyzed with regard to plasma levels of ACAN using ELISA. Samples of patients having a type A aortic dissection (group 1) also showed increased concentrations of ACAN compared to group 3. A significantly increased plasma concentration was detected in group 4 with patients having a surgical cryoablation, in which a desired severe atrial damage occurs intraoperatively. Particularly, group 4 (MAZE) showed an ACAN level which was about 9.9-fold increased compared to ctrl and about 2.1-fold increased compared to ao type A (FIG. 3). The increased ACAN concentrations in this group may be related to atrial damage, the use of the heart-lung machine during surgery, or the trauma of the operation itself.

### Example 3: Inter-Assay Comparison

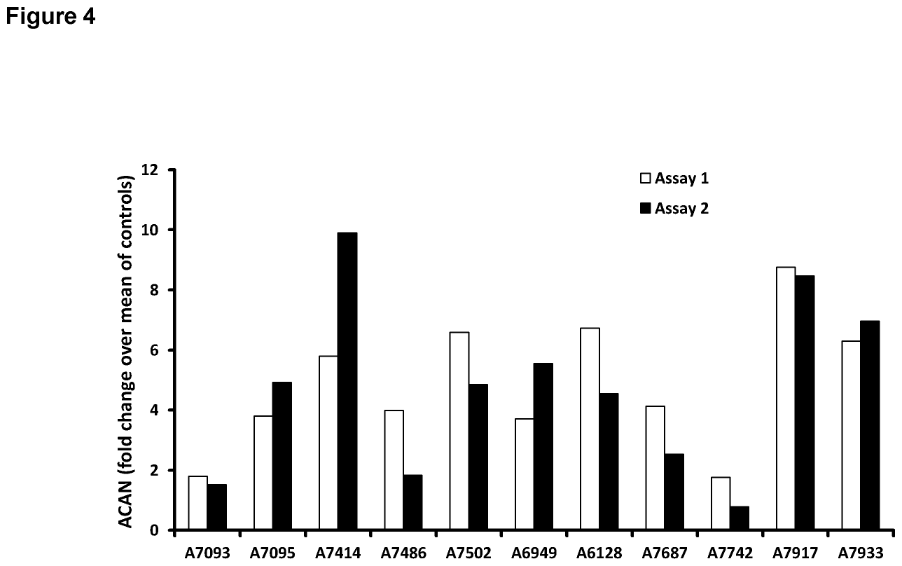

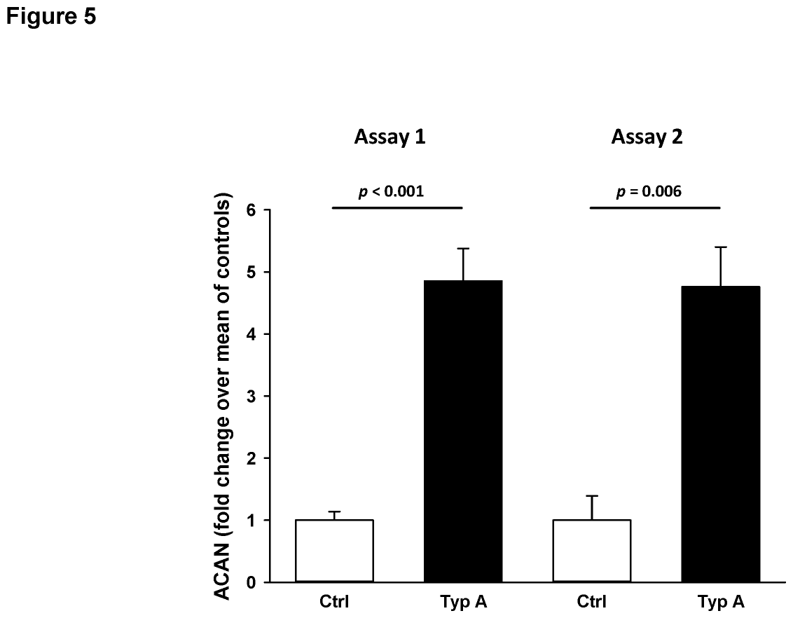

The repeated analyses of ACAN plasma concentrations using group 1 samples of eleven patients allow for a direct inter-assay comparison of experiment 1 and 2. FIG. 4 shows similar results for the increase in plasma concentrations of ACAN. Therefore, the ELISA kit used for performing the experiments allows for a reliable and reproducible determination of ACAN concentrations. FIG. 5 shows the mean values of experiments 1 and 2, i.e. the mean increase in ACAN plasma concentrations in group 1 compared to group 5.

### Example 4: Materials and Methods

### Blood Samples and Biopsies

Blood samples of ATAAD patients (n=33) were collected. Samples were drawn directly after admission to the hospital and centrifuged at 2,000×g for 10 min at 4° C. Plasma was partitioned in 200 μL aliquots and immediately stored at −80° C. within 30 min after admission until further use. Plasma samples from all other experimental cohorts were supplied by the German Heart Center Munich. Human biopsies (skeletal muscle, fat tissue, left atrium, aortic tissue from ATAAD or coronary artery bypass graft patients, Vena saphena magna and Arteria mammaria interna) were obtained during surgical procedures, directly snap-frozen and stored in liquid nitrogen until further use.

Assessment of Gene Expression in Human Biopsies by qRT-PCR

Frozen biopsies were homogenized in 900 μL QIAzol lysis reagent for 30 sec using an Ultraturrax MICCRA D-8 (ART Moderne Labortechnik, Müllheim, Germany) and processed with the RNeasy Plus UniversalMini Kit (QIAGEN, Hilden, Germany) according to the manufacturer's recommendation. One hundred ng total RNA were reverse-transcribed into cDNA with M-MLV reverse transcriptase (150 U, Invitrogen, Carlsbad, Calif.), random hexamer primers (375 ng), dNTPs (10 mM each), 10 mM DTT and 1× first strand buffer in a final volume of 30 μL for 50 minutes at 37° C. The enzyme was inactivated for 15 minutes at 70° C. Gene-specific amplification of 1 μL cDNA was performed on a Quant Studio 3 (ThermoFisher, Dreieich, Germany) with 0.3 μM of each primer and Power SYBR Green Mastermix (ThermoFisher) using the following cycling conditions: 95° for 10 minutes to activate Taq polymerase, followed by 40 cycles of 95° C. for 15 sec and 60° C. for 60 sec. The relative gene expression was normalized to ACTB (β-Actin) expression as the reference.

Measurement of ACAN, OGN, and ITGA11 in plasma samples by ELISA Commercially available ELISA kits were used to determine the concentration of aggrecan (ACAN) (Cat. No. SEB908Hu, Cloud Clone Corp., Katy, Tex.), osteoglycin (OGN) (Cat. No. LSF22608, LifeSpan Biosciences Inc., Seattle, Wash.) and integrin α 11 (ITGA11) (Cat. No. CSBEL011863HU, Cusabio, Houston, Tex.) in plasma samples according to the manufacturers' instructions. In brief, all components and samples were brought to room temperature and 100 μL of undiluted plasma samples were added, processed and plates were read at 450 nm. In each assay a standard curve was included to determine the concentration in individual samples.

### Statistical Analysis

Differences in gene expression were determined by the Mann-Whitney Rank Sum test or the one-way-ANOVA test. Significance of differences in ACAN protein concentrations for multiple groups was estimated by the Wilcoxon-Mann-Whitney, Kruskal Wallis or one-way-ANOVA test. In all cases a p value<0.05 was considered to be significant. Values are presented as mean±standard error of the mean (SEM), 95% confidence interval (CI) and fold change as appropriate.

### Example 5: Selection of Candidate Genes to Diagnose Acute Type a Aortic Dissection

The inventors previously identified 8,699 proteins in the aorta of the human heart. To limit the number of possible biomarker candidates, the inventors conducted two approaches. Firstly, the inventors selected proteins which were expressed most abundantly, but not necessarily restricted to aortic tissue. Secondly, the inventors selected proteins which were preferentially expressed in aortic tissue compared to all other fifteen heart regions.

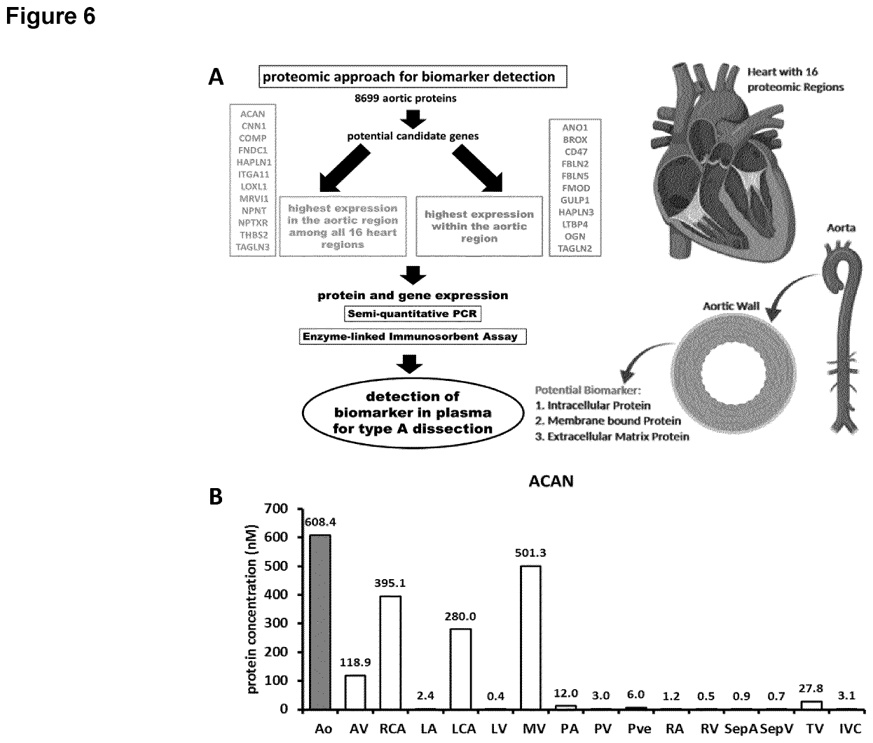

Following these pre-selection criteria, the inventors defined a list of 23 potential candidates (FIG. 6A). Looking at the protein expression of the candidate markers across the sixteen regions of the human heart, several candidates showed high expression in the aorta and coronary arteries, suggesting them as promising markers for vasculature. The concentration of ACAN protein, a promising candidate with a high specificity for arterial vessels across all sixteen regions of the human heart is shown in FIG. 6B. Next, the inventors determined mRNA expression of all candidates in aortic tissue from ATAAD patients in comparison to aortic tissue from coronary artery bypass patients. Furthermore, the inventors measured mRNA in left atrial tissue, venous and arterial vessels and the inventors analyzed the expression in extra-cardiac tissues such as fat and skeletal muscle. FIG. 1 shows the expression of four candidate genes: HAPLN1 (hyaluronan and proteoglycan link protein 1), ITGA11 (integrin α-11), OGN (osteoglycin) and ACAN (aggrecan). In all cases the mRNA abundance is highest in the aorta from ATAAD patients and it is significantly different to aortic tissue from coronary artery bypass graft patients (FIG. 1).

### Example 6: ACAN Protein Concentration is Enhanced in Plasma of Patients with Acute Type A Aortic Dissection

The data on protein and gene expression prompted the inventors to determine the protein concentration of ACAN, OGN and ITGA11 in plasma samples of ATAAD patients, obtained directly after the arrival at the hospital. For comparison, the inventors analyzed plasma of healthy volunteers and patients who underwent minimally invasive, isolated mitral valve repair (MVR). Indeed, ACAN levels were significantly elevated, with a four to five-fold higher concentration compared to both control groups (FIG. 2). Mean plasma ACAN level was 50.16±5.43 ng/mL. Mean plasma levels of the healthy subjects (control) and MVR group were 10.33±1.42 ng/mL and 11.92±1.77 ng/mL, respectively. The levels of OGN were also significantly enhanced in ATAAD samples with a mean value of 25.34±1.46 ng/mL compared to control and MVR samples with 17.65±2.58 ng/mL and 18.75±2.65 ng/mL, respectively. However, the difference between ATAAD patients and control groups was much smaller (FIG. 2). In contrast, ITGA11 values in plasma samples were lowest in the ATAAD group with 5.59±3.79 ng/mL and similar in the two reference groups (control and MVR) with 19.31±11.54 ng/μL and 22.84±17.88 ng/mL (FIG. 2). Thus, ACAN is the most promising candidate to diagnose ATAAD.

### Example 7: ACAN Plasma Levels are not Enhanced in Patients with Acute Myocardial Infarction and Aneurysm

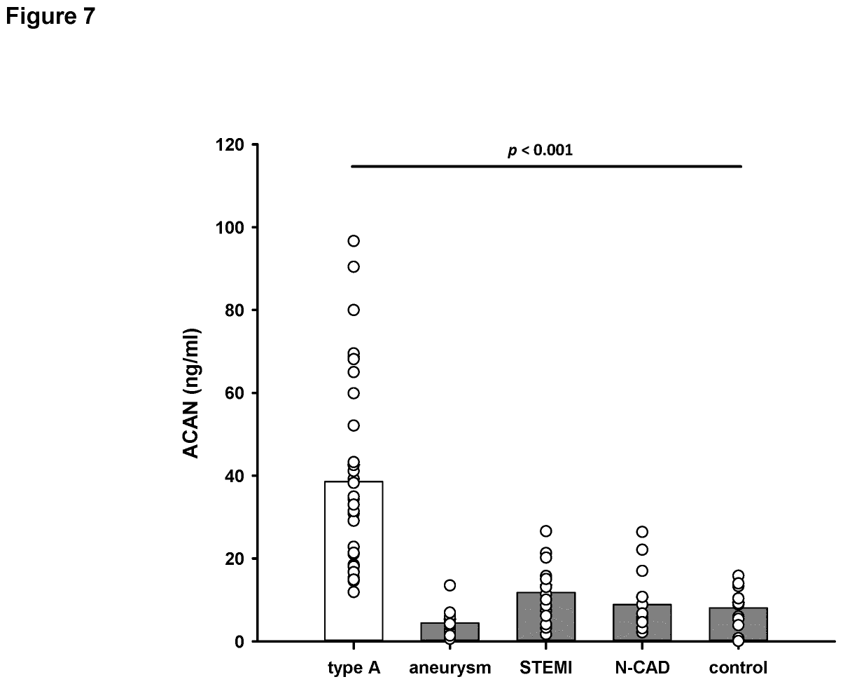

The inventors further addressed the question whether elevated ACAN plasma levels are specific for ATAAD. To further substantiate the initial promising results, the inventors increased the number of ATAAD patients (n=33). Using this extended cohort, the inventors detected a significant almost 10-fold increase in plasma levels of ACAN in ATAAD patients with a mean plasma level of 38.59±4.08 ng/mL compared to samples from patients with asymptomatic chronic aneurysm of the ascending aorta with a mean value of 4.45±0.90 ng/mL (FIG. 7). The inventors next analyzed ACAN plasma levels of patients with acute ST-elevation myocardial infarction (STEMI), which may confound the correct diagnosis of ATAAD. Again, ACAN protein concentrations of ATAAD patients were clearly and significantly elevated compared to STEMI patients who showed a mean value of 11.77±1.89 ng/mL (FIG. 7). In addition, ACAN protein levels in patients without coronary artery disease (N-CAD) are significantly lower compared to ATAAD patients, but not significantly different to healthy controls or STEMI patients. N-CAD group showed a mean value of 8.88±1.8 ng/mL (FIG. 7). Mean value of the healthy control group was 8.05±1.38 ng/mL. Thus, ACAN protein levels of ATAAD patients in the circulation are significantly elevated compared to healthy controls and patients with important cardiac differential diagnoses, including MI, supporting the use of ACAN as a reliable and specific biomarker to detect ATAAD.

### Example 8: Association of ACAN Plasma Concentration with Demographic Parameters and Severity of ATAAD

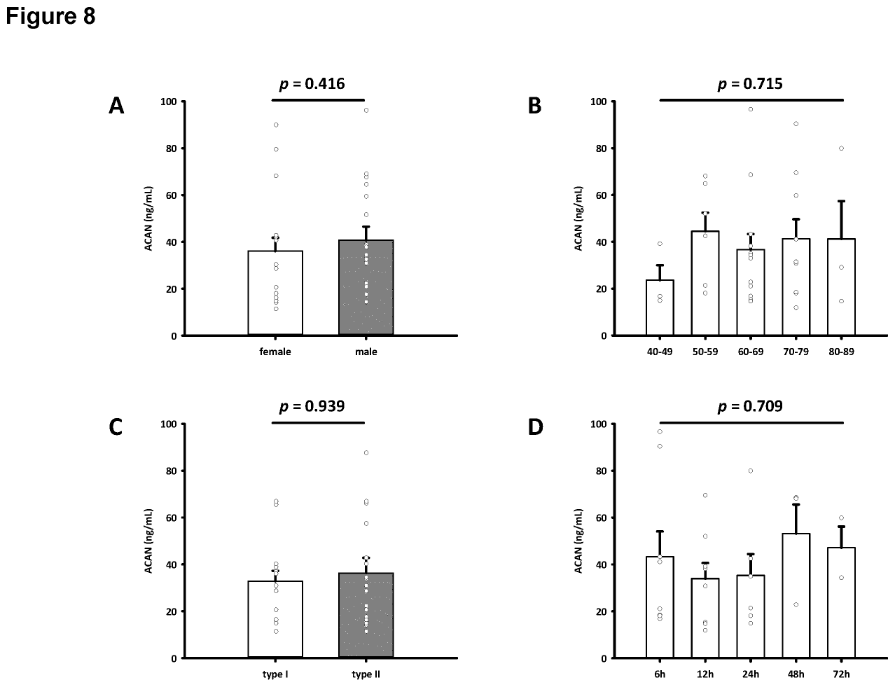

The inventors further addressed the question whether the release of ACAN into the circulation might be affected by basic demographic parameters such as sex or age. However, neither sex nor age had a significant impact on ACAN plasma levels (FIGS. 8A and B). Mean ACAN plasma levels of female and male samples were 36.06±5.74 ng/mL and 40.69±5.80 ng/mL. For age association, the ATAAD samples were divided into five age groups. Mean ACAN plasma levels of the five age groups, organized from young to old, were 23.60±6.38 ng/mL, 44.49±7.94 ng/mL, 36.63±6.69 ng/mL, 41.28±8.37 ng/mL and 41.21 ng/mL (FIG. 8B). Despite the considerably lower mean ACAN level of 23.60 ng/mL in the first group (40-49 years) compared to the mean ACAN levels of the other four age groups, there was no statistically significant difference of ACAN plasma levels between all five groups according to the one-way-ANOVA test with a p value of 0.715. Furthermore, the inventors considered whether extent of ATAAD, according to the De Bakey classification might be reflected by the ACAN concentration in plasma. However, there was no major difference between patients with De Bakey type I and II ATAAD (FIG. 8C) with mean ACAN levels of 32.79±4.43 ng/mL and 36.27±6.51 ng/mL. Finally, the inventors established kinetics of ACAN levels and the time period between onset of symptoms of ATAAD and the drawing of the blood samples. ACAN levels remained clearly elevated for up to 72 h after the onset without major differences at any time point (FIG. 8D). ACAN plasma levels of the five time points in increasing order were 43.2±10.84 ng/mL, 34.0±6.67 ng/mL, 35.3±9.06 ng/mL, 53.2±12.39 ng/mL and 47.1±9.03 ng/mL (p=0.709).

### Example 9: ACAN Detects Acute Type A Aortic Dissection with High Specificity and Sensitivity

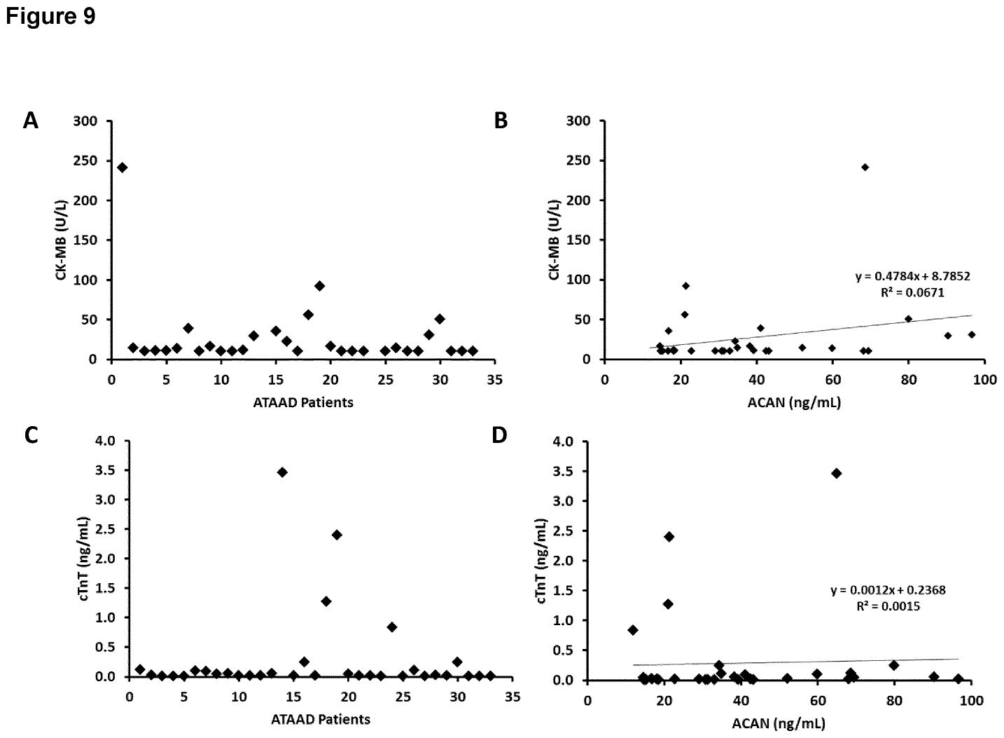

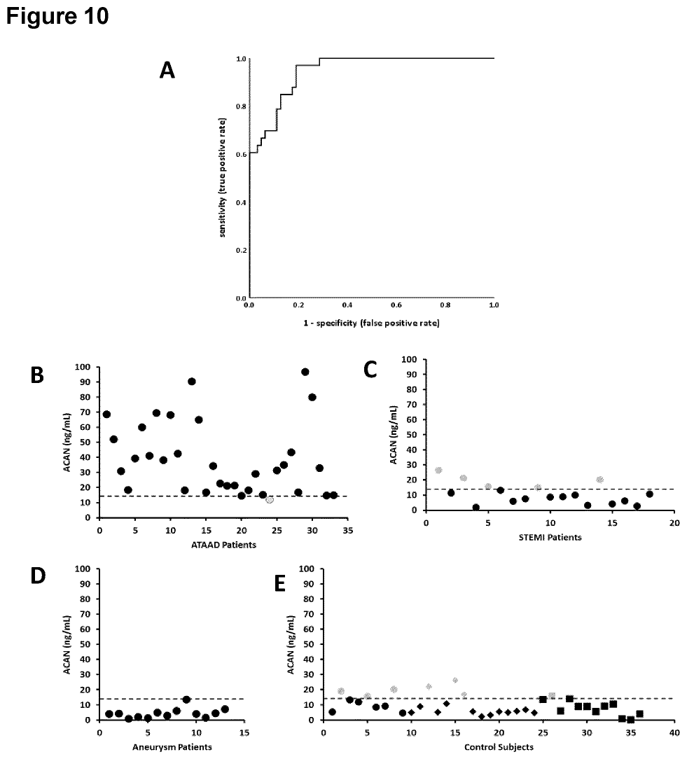

The inventors next evaluated the level of the clinical MI biomarkers, CK-MB and cTnT, in plasma samples of patients with ATAAD. For both markers, in the vast majority of samples, the values remained below the established clinical reference limit which defines myocardial cell damage (FIGS. 9A and C). In addition, no correlation between plasma levels of ACAN and CK-MB (FIG. 9B) or cTnT (FIG. 9D) was seen. All ATAAD samples with cardiac enzyme levels above the established clinical threshold suffered an involvement of the aortic root with presumably consecutive narrowing or obstruction of the coronary ostia. Thus, the increase of ACAN in the peripheral circulation of ATAAD patients apparently happens completely independent of both CK-MB and cTnT. Area under the curve on receiver-operator characteristics (ROC) curve analysis for all ATAAD patients (n=33) versus all control subjects (n=63) was 0.947 (FIG. 10A). Based on the ROC curve analysis an ACAN concentration of 14.3 ng/mL in the plasma was the optimum discrimination limit, resulting in a sensitivity of 97% and a specificity of 81%. Only one ATAAD sample showed an ACAN concentration below this threshold (FIG. 10B). Analyzing the ACAN levels in patients with cardiac complications (STEMI or aneurysms, FIGS. 10C and D) showed a specificity of more than 80%. In addition, in different experimental control groups (FIG. 10E) a similar specificity was obtained. Thus, the data clearly show the potential of ACAN as a reliable biomarker in plasma samples to detect ATAAD with a high sensitivity and specificity.

### Example 10: Discussion

ATAAD patients are often hospitalized with concomitant co-morbidities which mask and complicate the diagnosis of ATAAD, demanding a high specificity for a reliable biomarker. The inventors have measured ACAN levels in peripheral blood of ATAAD patients. The data clearly show that ACAN concentrations were significantly increased in plasma of ATAAD patients compared to plasma samples of healthy individuals and patients suffering from different cardiovascular disease. ACAN levels in ATAAD patients were elevated above the calculated threshold of 14.3 ng/mL based on the ROC curve analysis. In contrast, the ACAN plasma levels of the vast majority of patients with MI remained below this value. In addition, aneurysmatic alterations of the ascending thoracic aorta also did not result in increased ACAN plasma levels. Therefore, this study could definitely rule out ACAN as a possible screening marker for aneurysmatic thoracic aortic disease. In summary, it can therefore be said that secondary cardiovascular diagnoses did not influence the level of ACAN in plasma for specific diagnosis of ATAAD. Basic demographic parameters (age, sex), extent of the disease and the time between onset of ATAAD and hospitalization do not influence peripheral ACAN levels, suggesting that only the traumatic event of ATAAD would lead to ACAN release. Applying the optimum discrimination limit of 14.3 ng/mL, based on the ROC curve analyses, across the cohort of ATAAD patients, healthy probands and patients with other cardiovascular diagnoses (MVR, N-CAD) yielded a specificity of more than 97% and a sensitivity of 81% when considering all of the experimental control groups. Even when the inventors focused on clinical patients and excluded the healthy persons, the inventors still ended up with a sensitivity of >81%. Calponin and D-dimer have been proposed as diagnostic tools in ATAAD. Comparing the above results with these two markers the inventors found a superior specificity of ACAN to discriminate ATAAD and MI 73%). Importantly, ACAN levels did not correlate with CK-MB or cTnT concentrations. Thus, a combination of ACAN with these markers might be beneficial to further increase the sensitivity. Therefore, the combined use of ATAAD and MI markers in an emergency setting should prompt the treating physician to run the appropriate, more invasive, and time demanding diagnostic test for definitive confirmation. Thus, unnecessary therapeutic delays will be prevented. The level of calponin increases in ATAAD but decreases beyond 12 h after onset. In contrast, upon arrival at the hospital ACAN level stayed elevated and did not vary substantially for up to 72 h after the onset of ATAAD. This might especially be crucial when ATAAD occurred before that time period. Comparing the performance of ACAN with the existing markers, such as D-dimers and calponin, clearly underlines the superiority of ACAN.

In summary, the inventors have identified ACAN plasma levels as a reliable biomarker to detect the presence of an ATAAD. This marker reliably detected ATAAD patients in a very sensitive manner. At the same time, the biomarker showed a satisfying specificity which was not confounded by the presence of MI.

### REFERENCES

[1] Cikach et al., Massive aggrecan and versican accumulation in thoracic aortic aneurysm and dissection; JCI Insight. 2018; 3(5):e97167. https://doi.org/10.1172/jci.insight.97167.

The features disclosed in the specification, the claims, and/or in the accompanying figures may, both separately and in any combination thereof, be material for realizing the invention in various forms thereof.

1. A method of diagnosing an aortic dissection in a sample, wherein the method comprises:
a) providing a sample of a patient, wherein said sample is a blood sample, a serum sample, and/or a plasma sample;
measuring a level of aggrecan or a variant thereof; and
c) comparing the level(s) measured to a respective reference value and/or a reference sample of a healthy person not suffering from an aortic dissection.

2. The method according to claim 1,
further comprising determining that the patient has an aortic dissection, when the level of aggrecan in said sample is higher than the reference value and/or reference sample.

3. The method according to claim 1, wherein said aortic dissection is selected from a type A aortic dissection and a type B aortic dissection.

4. The method according to claim 1, wherein said sample is a human sample.

5. The method according to claim 1, further comprising measuring a level of at least one further biomarker selected from the group consisting of OGN, LPYD, ITGA11, ANO1, BROX, C10orf11, CD47, CNN1, COMP, FBLN2, FBLN5, FMOD, FNDC1, GULP1, HAPLN1, HAPLN2, LOXL1, LTBP4, MRVI1, MYH9, NPNT, NPTXR, SNAP23,TAGLN2, TAGLN3, THBS2, VCAN, variants thereof, and combinations thereof.

6. The method according to claim 1, wherein said measuring of the level of aggrecan or a variant thereof is performed by a method selected from the group comprising ELISA, PCR, qPCR, flow cytometry, mass spectrometry, antibody-based protein chips, 2-dimensional gel electrophoresis, western blot, protein immuno-precipitation, radio immunoassay, ligand binding assay, and liquid chromatography, preferably selected from ELISA, radio immunoassay, antibody-based protein chips, and combinations thereof.

7. The method according to claim 1, wherein said level refers to a level of protein and/or nucleic acid.

8. (canceled)

9. (canceled)

10. (canceled)

11. (canceled)

12. (canceled)

13. A kit for diagnosing an aortic dissection in a sample, wherein the kit comprises:
an agent for detecting aggrecan as a biomarker, wherein the agent comprises an antibody or an antigen-binding peptide;
a reference sample of a healthy person or a recombinant aggrecan in a defined amount;
optionally one or more agent(s) or means for detecting a biomarker other than aggrecan;
optionally auxiliary compounds for performing the method as defined in claim 1; and
optionally comprising instructions for comparing the aggrecan level measured to the reference sample of the healthy person not suffering from an aortic dissection, wherein an increase in the aggrecan level indicates an aortic dissection.

14. A point-of-care device for performing the method of diagnosing an aortic dissection as defined in claim 1, wherein the device comprises:
a sample inlet for contacting a sample selected from a blood sample, a serum sample, and/or a plasma sample with said point-of-care device;
an analyzing unit for measuring an aggrecan level in said sample; and
an evaluation unit comprising a detector for detecting an aggrecan level, wherein said detector is configured to generate an output signal indicating an aggrecan level.

15. The method according to claim 5, wherein the further biomarker is selected from the group consisting of OGN, LPYD, ITGA11, variants thereof, and combinations thereof.
